SEQUENCE LISTING (4 sequences)

SEQ ID NO: 1 - protein, 5 aa, Artificial Sequence
  Synthetic sequence
FMILL

SEQ ID NO: 2 - protein, 5 aa, Artificial Sequence
  Synthetic sequence
FRILL

SEQ ID NO: 3 - DNA, 15 bp, Artificial Sequence
  Synthetic sequence
ttcatgattttactc

SEQ ID NO: 4 - DNA, 15 bp, Artificial Sequence
  Synthetic sequence
ttcaggattttactc

(19) United States
(12) Patent Application Publication
(10) Pub. No.: US 2022/0193082 A1
(43) Pub. Date: Jun. 23, 2022

(54) COMBINATION TREATMENT OF SPECIFIC FORMS OF EPILEPSY

(71) Applicant: ZOGENIX INTERNATIONAL LIMITED, Berkshire (GB)

(72) Inventors: Peter De Witte, Kessel-Lo (BE); Lieven Lagae, Oud Heverlee (BE); Jo Sourbron, Bilzen (BE); Bradley S. Galer, West Chester, PA (US)

(73) Assignee: ZOGENIX INTERNATIONAL LIMITED, Berkshire (GB)

(21) Appl. No.: 17/579,135

(22) Filed: Jan. 19, 2022

Related U.S. Application Data
(63) Continuation of application No. 15/989,812, filed on May 25, 2018, now abandoned.
Application No. 15/989,812 is a continuation of application No. 15/265,062, filed on Sep. 14, 2016, now abandoned.
(60) Provisional application No. 62/218,456, filed on Sep. 14, 2015.

(30) Foreign Application Priority Data
Nov. 12, 2015 (JP) 2015-221719
Nov. 12, 2015 (JP) 2015-221721

(51) Int. Cl. A61K 31/536; A61K 31/55; A61K 31/475; A61K 45/06; A61K 31/343; A61K 31/19; A61K 31/551

(52) U.S. Cl. CPC A61K 31/536; A61K 31/55; A61K 31/475; A61K 31/551; A61K 31/343; A61K 31/19; A61K 45/06

(57) ABSTRACT

Formulations for and methods of treatment of Dravet syndrome that avoid side effects are disclosed. The formulations comprise a 5-HT receptor agonists which does not agonize selected 5-HT receptor subtypes, and in particular does not agonize the receptor subtype 5-HT2B. Also disclosed are combinations of such 5-HT receptor agonists. Also disclosed are combinations of such 5-HT receptor agonists and SSRIs, SNRIs, and triptans for treating co-morbidities associated with Dravet syndrome.

### FIELD OF THE INVENTION

The present invention relates to formulations and methods for the prevention of seizures and associated impairment resulting from Epilepsy and Dravet Syndrome.

### BACKGROUND OF THE INVENTION

Epilepsy is a condition of the brain marked by a susceptibility to recurrent seizures. There are numerous causes of epilepsy including, but not limited to birth trauma, perinatal infection, anoxia, infectious diseases, ingestion of toxins, tumors of the brain, inherited disorders or degenerative disease, head injury or trauma, metabolic disorders, cerebrovascular accident and alcohol withdrawal.

A large number of subtypes of epilepsy have been characterized and categorized. The most recent classification and categorization system, and the one that is widely accepted in the art, is that adopted by the International League Against Epilepsy's (“ILAE”) Commission on Classification and Terminology [See e.g., Berg et al., “Revised terminology and concepts for organization of seizures,” Epilepsia, 51(4):676-685 (2010)]:
I. ELECTROCHEMICAL SYNDROMES (arranged by age of onset):
A. Neonatal period
1. Benign familial neonatal epilepsy (BFNE)
2. Early myoclonic encephalopathy (EME)
3. Ohtahara syndrome
B. Infancy
1. Epilepsy of infancy with migrating focal seizures
2. West syndrome
3. Myoclonic epilepsy in infancy (MEI)
4. Benign infantile epilepsy
5. Benign familial infantile epilepsy
6. Dravet syndrome
7. Myoclonic encephalopathy in non-progressive disorders
C. Childhood
1. Febrile seizures plus (FS+) (can start in infancy)
2. Panayiotopoulos syndrome
3. Epilepsy with myoclonic atonic (previously astatic) seizures
4. Benign epilepsy with centrotemporal spikes (BECTS)
5. Autosomal-dominant nocturnal frontal lobe epilepsy (ADNFLE)
6. Late onset childhood occipital epilepsy (Gastaut type)
7. Epilepsy with myoclonic absences
8. Lennox-Gastaut syndrome
9. Epileptic encephalopathy with continuous spike-and-wave during sleep (CSWS), also known as Electrical Status Epilepticus during Slow Sleep (ESES)
10. Landau-Kleffner syndrome (LKS)
11. Childhood absence epilepsy (CAE)
D. Adolescence—Adult
1. Juvenile absence epilepsy (JAE)
2. Juvenile myoclonic epilepsy (JME)
3. Epilepsy with generalized tonic—clonic seizures alone
4. Progressive myoclonus epilepsies (PME)
5. Autosomal dominant epilepsy with auditory features (ADEAF)
6. Other familial temporal lobe epilepsies
E. Less specific age relationship
1. Familial focal epilepsy with variable foci (childhood to adult)
2. Reflex epilepsies
II. DISTINCTIVE CONSTELLATIONS
A. Mesial temporal lobe epilepsy with hippocampal sclerosis (MTLE with HS)
B. Rasmussen syndrome
C. Gelastic seizures with hypothalamic hamartoma
D. Hemiconvulsion—hemiplegia—epilepsy
E. Epilepsies that do not fit into any of these diagnostic categories, distinguished on the basis of
1. Presumed cause (presence or absence of a known structural or metabolic condition)
2. Primary mode of seizure onset (generalized vs. focal)
III. EPILEPSIES ATTRIBUTED TO AND ORGANIZED BY STRUCTURAL-METABOLIC CAUSES
A. Malformations of cortical development (hemimegalencephaly, heterotopias, etc.)
B. Neurocutaneous syndromes (tuberous sclerosis complex, Sturge-Weber, etc.)
C. Tumor
D. Infection
E. Trauma
IV. ANGIOMA
A. Perinatal insults
B. Stroke
C. Other causes
V. EPILEPSIES OF UNKNOWN CAUSE
VI. CONDITIONS WITH EPILEPTIC SEIZURES NOT TRADITIONALLY DIAGNOSED AS FORMS OF EPILEPSY PER SE
A. Benign neonatal seizures (BNS)
B. Febrile seizures (FS)

Note that the foregoing arrangement of electroclinical syndromes does not reflect etiology.

Those skilled in the art will recognize that these subtypes of epilepsy are triggered by different stimuli, are controlled by different biological pathways and have different causes, whether genetic or environmental. In other words, the skilled artisan will recognize that teachings relating to one epileptic subtype are not necessarily applicable to other subtypes. Specifically, different epilepsy subtypes respond differently to different anticonvulsant drugs.

There are a large number of different drugs which have been used in the treatment of various forms of epilepsy. Although the list below is not comprehensive, it is believed to include those drugs which are widely prescribed in patients diagnosed with epilepsy.
Carbatrol, Epitol, Equetro, TEGretol (carbamazepine)
Gabitril (tiagabine)
Keppra (levetiracetam)
LaMICtal (lamotrigine).
Lyrica (pregabalin)
Gralise, Horizant, Neurontin, Gabarone (gabapentin)
Dilantin, Prompt, Di-Phen, Epanutin, Phenytek (phenytoin)
Topamax, Qudexy XR, Trokendi XR, Topiragen (topiramate)
Trileptal, Oxtellar (oxcarbazepine)
Depacon, Depakene, Depakote, Stavzor (valproate, valproic acid)
Zonegran (zonisamide)
Fycompa (perampanel)
Aptiom (eslicarbazepine acetate)
Vimpat (lacosamide)
Sabril (vigabatrin)
Banzel, Inovelon (rufinamide)
Cerebyx (fosphenytoin)
Zarontin (ethosuximide)
Solfoton, Luminal (phenobarbital)
Valium, Diastat (diazepam),
Ativan (lorazepam)
Lonopin, Klonopin (clonazepam)
Frisium, Onfi (clobazam)
Potiga (ezogabine)
Felbatol (felbamate)
Mysoline (primidone)

Thus, there are a large number of different drugs which have been used in the treatment of various forms of epilepsy, and different epilepsy subtypes respond differently to different anticonvulsant drugs. Thus, persons of ordinary skill in the art recognize that whether a patient with a particular type of epilepsy will respond to a particular drug is not predictable, and hence the efficacy of a particular drug for a particularly type of epilepsy is a surprising result.

Dravet Syndrome is a rare and catastrophic form of intractable epilepsy that begins in infancy. Initially, the patient experiences prolonged seizures. In their second year, additional types of seizure begin to occur and this typically coincides with a developmental decline, possibly due to repeated cerebral hypoxia. This leads to poor development of language and motor skills.

Children with Dravet Syndrome are likely to experience multiple seizures per day. Epileptic seizures are far more likely to result in death in sufferers of Dravet Syndrome; approximately 10 to 15% of patients diagnosed with Dravet Syndrome die in childhood, particularly between two and four years of age. Additionally, patients are at risk of numerous associated conditions including orthopedic developmental issues, impaired growth and chronic infections.

The cost of care for Dravet Syndrome patients is also high as the affected children require constant supervision and many require institutionalization as they reach teenage years.

The presentation and diagnosis of Dravet syndrome differs significantly from other forms of epilepsy. The Ceulemans (2011) article states that Dravet syndrome can be distinguished from other forms of epilepsy by:

“ . . . the appearance of tonic-clonic seizures during the first year of life, the occurrence of myoclonic seizures and ataxia later, impaired psychomotor development following the onset of the seizures, and poor response to anti-epileptic drugs.”

This is supported further by the Brunklaus et al. article which states the following:

“Dravet syndrome typically presents in the first year of life with prolonged, febrile and afebrile, generalized clonic or hemiclonic epileptic seizures in children with no pre-existing developmental problems. Other seizure types including myoclonic, focal and atypical absence seizures appear between the ages of 1 and 4 years (Dravet, 1978).”

Thus, the presentation and diagnosis of Dravet syndrome is significantly different from other forms of epilepsy. One of ordinary skill in the art would not find it obvious or assume that any particular compound would be efficacious in Dravet syndrome.

It is known in the art (Ceulemans (Developmental Medicine & Child Neurology, 2011, 53, 19-23, PTO-892, Brunklaus et al. (BRAIN, 2012, pages 1-8, PTO-892) that mutations in the alpha-subunit of the neuron-specific voltage-gated sodium channel (SCN1a) was discovered as the primary genetic cause for Dravet syndrome in 2001. Thus, the cause of Dravet syndrome is significantly different as compared to other forms of epilepsy. Unlike other forms of epilepsy, diagnosis of Dravet is based in part on detection of these genetic mutations in addition to clinical observation. Consequently, there has been an increase in the number of patients diagnosed with the disease.

Of particular concern, children with Dravet Syndrome are particularly susceptible to episodes of Status Epilepicus. This severe and intractable condition is categorized as a medical emergency requiring immediate medical intervention, typically involving hosptialization. Status Epilepticus can be fatal. It can also be associated with cerebral hypoxia, possibly leading to damage to brain tissue. Frequent hospitalizations of children with Dravet Syndrome are clearly distressing, not only to the patient but also to family and care givers.

At present, although a number of anticonvulsant therapies can be employed to reduce the instance of seizures in patients with Dravet Syndrome, the results obtained with such therapies are typically poor and those therapies only affect partial cessation of seizures at best. Seizures associated with Dravet Syndrome are typically resistant to conventional treatments. Further, many anticonvulsants such as clobazam and clonazepam have undesirable side effects, which are particularly acute in pediatric patients.

Additionally, as mentioned in the excerpt above from Ceulemans (2011), prior to the current invention Dravet syndrome was believed to be refractory to treatment with all existing epilepsy drugs, leading to unavoidable permanent impairment. Ceulemans additionally states:

“Most often, parents are distraught in view of these sudden frightening convulsions, and the first impression they have is that their child is dying. That leads them to rush to the nearest emergency department where staff physicians manage the seizures, which are long-lasting, drug-resistant and require higher doses (emphasis added) of benzodiazepines than usual to stop them.”

This is supported further by Brunklaus et al., which states the following:

“The epilepsy is usually refractory to standard anti-epileptic medication (emphasis added) and from the second year of life affected children develop an epileptic encephalopathy resulting in cognitive, behavior and motor impairment”

In fact, before the current invention it had been found that add on drug treatment using current epilepsy drugs resulted in a 50% decrease in seizure frequency in only 20-30% of patients will, and less than 5% became seizure free. The Ceulemans 2011 paper also cautions against expecting that a Dravet syndrome patient will become seizure free, stating:

“It is understandable that parents want their children to be seizure-free, but they should be informed that it is probably an unattainable goal in this highly drug-resistant syndrome (emphasis added)” (at page 21, col. 1)

In addition, it has been found that a certain class of drugs that are widely used in treating epilepsy, namely sodium channel blockers including carbamazepine, oxcarbazepine, lamotrigine, lacosamide, rufinamide, phenytoin, and fosphenytoin are contra-indicated in Dravet syndrome. These drugs have been found to lead to a greater incidence of seizures in almost all Dravet syndrome patients. Similarly, selective GABA reuptake inhibitors/GABA T inhibitors including vigabatrin and tiagabine should be avoided in Dravet syndrome.

Sodium channel blockers preferentially affect the sodium channel at a specific stage of its cycle of rest, activation and inactivation, often by delaying the recovery from the inactivated state, thereby producing a cumulative reduction of Na+.

Non-epileptic brains have a natural balance of excitation (that can evoke seizures) and inhibition (that can reduce seizures). In epilepsies that are caused by too much excitatory neurotransmission, sodium channel blockers are beneficial because they reduce the neurotransmitters that cause too much excitation.

In contrast, patients with Dravet syndrome have gene mutations, such as SCN1A mutation, which cause a loss of sodium channel function. Based on the mechanism in which sodium channel blockers work to prevent seizure activity, one would think that these mutations that cause the sodium channel to be ineffective (in essence, blocked) should prevent seizures and make a person with Dravet syndrome less prone to epilepsy. However, this loss of function in fact leads to increased seizure activity because the result of this mutation is a decreased amount of inhibitory neurotransmitter that normally exists in the correct amount in the brain to balance excitatory neurotransmitters that make seizure more likely to occur. In this situation, the problem with the balance of excitation and inhibition in the brain is not too much excitation, it is too little inhibition. Giving sodium channel blocking drugs to Dravet syndrome patients further decreases the amount of inhibitory neurotransmitters in the brain, tipping the balance toward more seizure activity.

In Arzimanoglou (Epilepsia, 50 (Suppl. 8):3-9, 2009) it is stated:

“Many AEDs have no effect and may be at the origin of adverse effects, such as carbamazepine and vigabatrin which can favor or even induce myoclonic seizures and lamotrigine, particularly for young patients.”

In Chiron et. al. (Epilepsia, 52 (Suppl. 2):72-75, 2011) it is stated:

“Soon after the identification of the syndrome, compounds that worsened symptoms were identified, namely lamotrigine that involves up to 80% of the patients . . . ; carbamazepine and vigabatrin worsening is in the order of 60%. Lamotrigine, (and) carbamazepine . . . should be avoided because they may worsen seizures.”

In summary, there are many different drugs used to treat epilepsy, many of which were ineffective or which exacerbated symptoms. Thus, as shown above Dravet syndrome was believed to be drug resistant.

Therefore, there is a dire, long-felt and previously unmet need for therapeutic agents which are effective in reducing seizures in epileptic patients diagnosed with Dravet syndrome.

### SUMMARY OF THE INVENTION

A method is disclosed for reducing seizures and/or related symptoms connected with epilepsy and/or Dravet syndrome comprising administering to a patient therapeutically effective amount of formulation comprising a pharmaceutically acceptable carrier and a 5-HT receptor agonist wherein the 5-HT receptor agonist does not recognize the 5-HT2B receptor subtype.

An aspect of the invention is the method of treatment as described herein wherein the 5-HT receptor agonist is selected from the group consisting of 3-[3-(2-Dimethylaminoethyl)-1H-indol-5-yl]-N-(4-methoxybenzyl)acrylamide, (4-Bromo-3,6-dimethoxybenzocyclobuten-1-yl)methylamine hydrobromide, (6aR,9R)—N-((2R,5S,10aS,10bS)-5-benzyl-10b-hydroxy-2-methyl-3,6-dioxooctahydro-2H-oxazolo[3,2-a]pyrrolo[2,1-c]pyrazin-2-yl)-7-methyl-4,6,6a,7,8,9-hexahydroindolo[4,3-fg]quinoline-9-carboxamide, (2S)-(+)-5-(1,3,5-Trimethylpyrazol-4-yl)-2-(dimethylamino)tetralin, 1H-Indol-5-ol, 3-(1-methyl-4-piperidinyl) and acids, bases, amines, salts, derivatives, fragments, and complexes thereof as well as any combination thereof.

Another aspect of the invention is a method of treatment as described herein wherein the 5-HT receptor agonist is lorcaserin [(1R)-8-chloro-1-methyl-2,3,4,5-tetrahydro-1H-3-benzazepine].

Another aspect of the invention is a method of treatment as described herein wherein the 5-HT receptor agonist is lisuride [3-[(6aR,9S)-7-methyl-6,6a,8,9-tetrahydro-4H-indolo[4,3-fg]quinoline-9-yl]-1,1-diethylurea].

Another aspect of the invention is a method of treatment as described herein wherein the 5-HT receptor agonist is efavirenz [(4S)-6-chloro-4-(2-cyclopropylethynyl)-4-(trifluoromethyl)-1H-3,1-benzoxazin-2-one].

Another aspect of the invention is a method of treatment as described herein wherein the 5HT receptor agonist is administered in combination with a second agent selected from the group consisting of 5-HT2B inhibitors, SSRIs, SNRIs, and triptans.

As shown above and as will be recognized by others skilled in the art the use of some drugs known to be useful in the treatment of epilepsy are actually harmful in the treatment of patients with Dravet syndrome. In view of such and further in view of the conventional wisdom that Dravet syndrome was generally not treatable with drugs, an efficacious compound for the treatment of Dravet syndrome would provide improved and unexpected results.

Ceulemans et. al. (Epilepsia, 53(7):1131-1139, 2012) discloses the use of fenfluramine in the treatment of Dravet syndrome, and discloses the result that when patients were subjected to long term treatment with fenfluramine in an amount of 0.12-0.90 mg/kg/day, 70% of patients were seizure-free, a useful and unexpected result, offering for the first time an efficacious treatment option for sufferers of Dravet syndrome. Fenfluramine is a potent 5-hydroxytryptamine (5-HT, serotonin) releaser that activates multiple 5-HTsubtype receptors. It is currently believed that treatment with fenfluramine in an amount ranging from 0.1-1.7 mg/kg/day or higher is effective in reducing or eliminating seizures and associated cognitive decline in Dravet syndrome.

5-HT receptors are a group of G protein-coupled receptors (GPCRs) and ligand-gated ion channels (LGICs) found in the central and peripheral nervous systems. They mediate both excitatory and inhibitory neurotransmission. The serotonin receptors are activated by the neurotransmitter serotonin, which acts as their natural ligand.

5-HT receptors modulate the release of many neurotransmitters, including glutamate, GABA, dopamine, epinephrine/norepinephrine, and acetylcholine, as well as many hormones, including oxytocin, prolactin, vasopressin, cortisol, corticotropin, and substance P, among others. They influence various biological and neurological processes such as aggression, anxiety, appetite, cognition, learning, memory, mood, nausea, sleep, and thermoregulation.

There are multiple 5-HTsubtype receptors, 14 of which have been described in humans, each of which are distributed in various organs and have multiple functions. These subtype receptors include 5-HT1A, 5-HT1B, 5-HT1D, 5-HT1E, 5-HT1F, 5-HT2A, 5-HT2B, 5-HT2C, 5-HT3, 5-HT4, 5-HT5A, 5-HT5B, 5-HT6, and 5-HT7.

Fenfluramine was known to have high affinity for and activity at the 5-HT2A, 5-HT2B and 5-HT2C receptor subtypes (Rothman et al, 2015). 5-HT2C-agonists trigger appetite suppression, and therefore fenfluramine has been applied for treating obesity, as part of the popular weight loss drug Fen-Phen (fenfluramine/phentermine). However, the activation of 5-HT2B receptors is associated with cardiac valve hypertrophy, and this drug-induced valvulopathy has resulted in the withdrawal of Fen-Phen from the market in September of 1997.

Thus, there is an un-met medical need for a 5-HTreceptor agonist with affinity to one or more 5-HT receptor subtypes with activity in Dravet syndrome, and with sufficient specificity to avoid the side effects such as cardiac valve hypertrophy associated with other 5-HT subtypes; and in particular a use of such an agonist in the treatment of forms of epilepsy and to relieve symptoms of Dravet syndrome.

The present invention meets that need.

It is an object of the invention to provide a compound and/or formulation as well as a method of use of such for the treatment of seizures in patients with epilepsy.

It is a further object of the invention to provide a compound and/or formulation as well as a method of use of such for the treatment of seizures in patients with Dravet syndrome.

It is a further object of the invention to provide a 5-HT receptor agonist and formulation as well as a method of use of such for the treatment of seizures in patients with epilepsy including Dravet syndrome.

It is a further object of the invention to provide one or more 5-HT receptor agonists with affinity to one or more 5-HT receptors which are effective in reducing seizures in patients with epilepsy; and in reducing seizures in patients with epilepsy and Dravet syndrome.

It is a further object of the invention to provide 5-HT receptor agonists effective in reducing seizures in patients with epilepsy, including Dravet syndrome, without affinity to one or more 5-HT receptor subtypes associated with side effects.

It is a further object of the invention to provide 5-HT receptor agonists effective in reducing seizures in patients with epilepsy, including Dravet syndrome, which are antagonists of one or more 5-HT receptor subtypes associated with side effects.

It is a further object of the invention to provide a 5-HT receptor agonist with affinity for one or more of 5-HT1A, 5-HT1B, 5-HT1D, 5-HT1E, 5-HT1F, 5-HT2A, 5-HT2B, 5-HT2C, 5-HT3, 5-HT4, 5-HT5A, 5-HT5B, 5-HT6, and 5-HT7, preferably for one or more of 5-HT1D, 5-HT1E, 5-HT2A, 5-HT2C, 5-HT5A, 5-HT5B, and 5-HT7, more preferably for one or more of 5-HT1D, 5-HT2A, 5-HT2C, 5-HT5A, 5-HT5B, 5-HT7, still more preferably for one or more of 5-HT2A or 5-HT2C. In a preferred embodiment, the 5-HT receptor agonist has affinity for the 5-HT2A receptor subtype, and in a particularly preferred embodiment, the 5-HT receptor agonist has high specificity for the 5-HT2A receptor subtype. In another preferred embodiment, the 5-HT receptor agonist has affinity for the 5-HT2C receptor subtype, and in a particularly preferred embodiment, the 5-HT receptor agonist has high specificity for the 5-HT2C receptor subtype. In another preferred embodiment, the 5-HT receptor agonist has affinity for the 5-HT2A and the 5-HT2C receptor subtype. In a particularly preferred embodiment, the 5-HT receptor is efavirenz.

It is a further object of the invention to supply a 5-HT receptor agonist with sufficient specificity to avoid agonizing those 5-HT receptors associated with undesired side effects. Preferably, 5-HT receptor agonist is not an agonist of one or more of 5-HT1A, 5-HT1B, 5-HT1D, 5-HT1E, 5-HT1F, 5-HT2A, 5-HT2B, 5-HT2C, 5-HT3, 5-HT4, 5-HT5A, 5-HT5B, 5-HT6, and 5-HT7, preferably of one or more 5-HT1A, 5-HT1B, 5-HT1E, 5-HT1F, 5-HT2B, 5-HT2C, 5-HT3, 5-HT4, and 5-HT6, more preferably of one or more of 5-HT1A, 5-HT1B, 5-HT1F, 5-HT2B, 5-HT3, 5-HT4, and 5-HT6. It is particularly preferred that the 5-HT receptor agonist does not agonize the 5-HT2B receptor subtype associated with cardiotoxic effects including valvulopathies. It is also particularly preferred that the 5-HT receptor agonist does not elicit hallucinogenic effects sometimes associated with activation of the 5-HT2A receptor subtype. In a preferred embodiment, the 5-HT receptor agonist does not agonize the 5-HT2A receptor. In another preferred embodiment, the 5-HT receptor agonist has high specificity to the 5-HT2C subtype relative to the 5-HT2A receptor subtype. In a particularly preferred embodiment, the 5-HT agonist is Lorcaserin,

It is a further object of the invention to supply a 5-HT receptor which is an antagonist of one or more of those 5-HT receptors associated with unwanted or potentially dangerous side effects. Preferably, 5-HT receptor agonist is an antagonist of one or more of 5-HT1A, 5-HT1B, 5-HT1D, 5-HT1E, 5-HT1F, 5-HT2A, 5-HT2B, 5-HT2C, 5-HT3, 5-HT4, 5-HT5A, 5-HT5B, 5-HT6, and 5-HT7, preferably of one or more 5-HT1A, 5-HT1B, 5-HT1E, 5-HT1F, 5-HT2B, 5-HT2C, 5-HT3, 5-HT4, and 5-HT6, more preferably of one or more of 5-HT1A, 5-HT1B, 5-HT1F, 5-HT2B, 5-HT3, 5-HT4, and 5-HT6. It is particularly preferred that the 5-HT receptor agonist is also an antagonist of the 5-HT2B receptor subtype, in order to reduce or eliminate side cardiovascular-related side effects. In a particularly preferred embodiment, the 5-HT agonist is lisuride.

Preferred receptor agonists include one or more of

GR 46611 [3-[3-(2-Dimethylaminoethyl)-1H-indol-5-yl]-N-(4-methoxybenzyl)acrylamide],

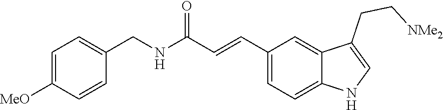 [SMILES: COc1ccc(CNC(=O)/C=C/c2ccc3[nH]cc(CCN(C)C)c3c2)cc1]

BRL 54443 [1H-Indol-5-ol, 3-(1-methyl-4-piperidinyl)-],

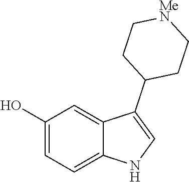 [SMILES: CN1CCC(c2c[nH]c3ccc(O)cc23)CC1]

TCB 2 [(4-Bromo-3,6-dimethoxybenzocyclobuten-1-yl)methylamine hydrobromide],

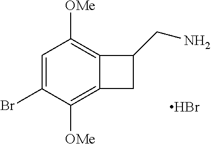 [SMILES: COc1cc(Br)c(OC)c2c1C(CN)C2 ; Br]

lorcaserin [(1R)-8-chloro-1-methyl-2,3,4,5-tetrahydro-1H-3-benzazepine],

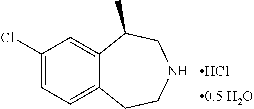

ergotamine [(6aR,9R)—N-((2R,5S,10aS,10bS)-5-benzyl-10b-hydroxy-2-methyl-3,6-dioxooctahydro-2H-oxazolo[3,2-a] pyrrolo[2,1-c]pyrazin-2-yl)-7-methyl-4,6,6a,7,8,9-hexahydroindolo[4,3-fg] quinoline-9-carboxamide],

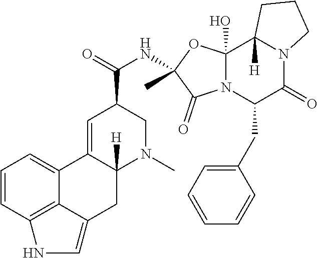

AS 19 [(2S)-(+)-5-(1,3,5-Trimethylpyrazol-4-yl)-2-(dimethylamino)tetralin],

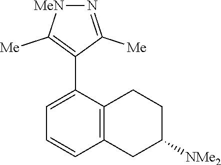 [SMILES: Cc1nn(C)c(C)c1-c1cccc2c1CC[C@H](N(C)C)C2]

Efavirenz [(4S)-6-chloro-4-(2-cyclopropylethynyl)-4-(trifluoromethyl)-1H-3,1-benzoxazin-2-one],

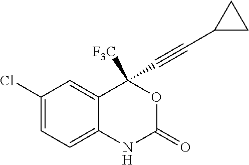

Lisuride [3-[(6aR,9S)-7-methyl-6,6a,8,9-tetrahydro-4H-indolo[4,3-fg]quinoline-9-yl]-1,1-diethylurea],

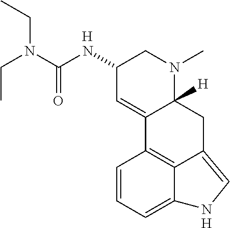

and salts, derivatives, fragments, and complexes thereof.

Particularly preferred is lorcaserin, due to its 100× affinity for the 5-HT2C receptor vs. the 5-HT2B. Thus, lorcaserin is expected to have similar efficacy to fenfluramine in epilepsy including Dravet syndrome, without the cardiovascular side effects including valvulopathy associated with the 5-HT2B receptor subtype. Further, the use of Lorcaserin is not associated with hallucinogenic effects that are mediated by activation of the 5-HT2A receptor subtype.

Also particularly preferred is efavirenz due to its activity and affinity for both the 5-HT2A and the 5-HT2C receptor. Thus, efavirenz is expected to have similar efficacy to fenfluramine in epilepsy including Dravet syndrome, without the cardiovascular side effects including valvulopathy.

Also particularly preferred is lisuride due to its dual agonist activity and affinity for the 5-HT2A receptor and antagonist activity at the 5-HT2B receptor. Thus, lisuride is expected to have similar efficacy to fenfluramine in epilepsy including Dravet syndrome, without the cardiovascular side including valvulopathy.

It is a further object of the invention to supply the 5-HT receptor agonist as described above in combination with one or more 5-HT receptor antagonist which are antagonists to one or more 5-HT receptor subtypes. Preferably the 5-HT receptor subtypes are one or more of 5-HT1A, 5-HT1B, 5-HT1D, 5-HT1E, 5-HT1F, 5-HT2A, 5-HT2B, 5-HT2C, 5-HT3, 5-HT4, 5-HT5A, 5-HT5B, 5-HT6, and 5-HT7, preferably one or more 5-HT1A, 5-HT1B, 5-HT1E, 5-HT1F, 5-HT2B, 5-HT2C, 5-HT3, 5-HT4, and 5-HT6, more preferably one or more of 5-HT1A, 5-HT1B, 5-HT1F, 5-HT2B, 5-HT3, 5-HT4, and 5-HT6. It is particularly preferred that the 5-HT receptor antagonist is an antagonist of the 5-HT2B receptor subtype. Preferred 5-HT2B receptor antagonists include but are not limited to

ATC 0175 [N-[cis-4-[[4-(Dimethylamino)-2-quinazolinyl]amino]cyclohexyl]-3,4-difluorobenzamide hydrochloride],

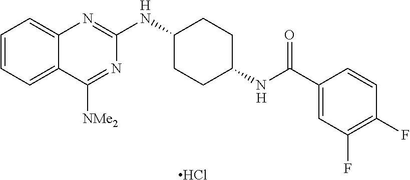

LY 266097 [1-[(2-Chloro-3,4-dimethoxyphenyl)methyl]-2,3,4,9-tetrahydro-6-methyl-1H-pyrido [3,4-b] indole hydrochloride],

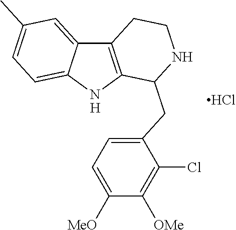

LY 272015 [1-[(3,4-Dimethoxyphenyl)methy]-2,3,4,9-tetrahydro-6-methyl-1H-pyrido[3,4-b]indole hydrochloride],

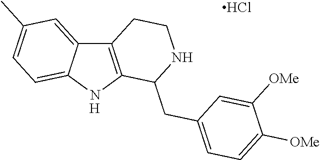

RS 127445 [4-(4-Fluoro-1-naphthalenyl)-6-(1-methylethyl)-2-pyrimidinamine hydrochloride],

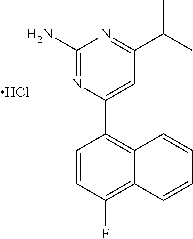 [SMILES: Cl ; CC(C)c1cc(-c2ccc(F)c3ccccc23)nc(N)n1]

SB 200646 [N-(1-Methyl-1H-indol-5-yl)-N′-3-pyridinylurea],

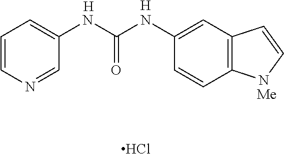

SB 204741 [N-(1-Methyl-1H-indolyl-5-yl)-N″-(3-methyl-5-isothiazolyl)urea],

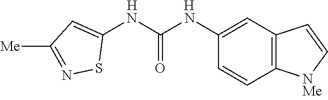

SB 206553 [3,5-Dihydro-5-methyl-N-3-pyridinylbenzo[1,2-b:4,5-b′]dipyrrole-1(2H)-carboxamide hydrochloride],

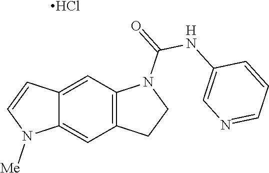

SB 221284 [2,3-Dihydro-5-(methylthio)-N-3-pyridinyl-6-(trifluoromethyl)-1H-indole-1-carboxamide],

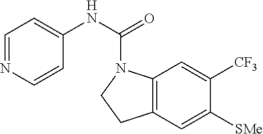

SB 228357 [N-[3-Fluoro-5-(3-pyrindyl)phenyl]-2,3-dihydro-5-methoxy-6-(trifluoromethyl)-1H-indole-1-carboxamide],

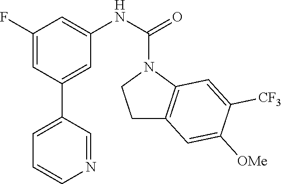

SDZ SER 082 [(+)-cis-4,5,7a,8,9,10,11,11a-Octahydro-7H-10-methylindolo[1,7-bc][2,6]-naphthyridine fumarate],

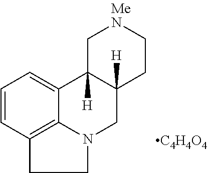

Lisuride [3-[(6aR,9S)-7-methyl-6,6a,8,9-tetrahydro-4H-indolo[4,3-fg]quinoline-9-yl]-1,1-diethylurea],

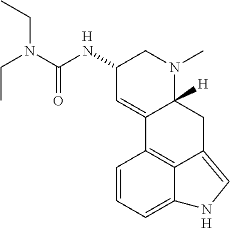

and combinations, salts, derivatives, fragments, and complexes thereof.

It is a further object of the invention to supply a method of treatment for epilepsy comprising delivering one or more of the above 5-HT receptor agonists to a patient suffering from epilepsy; and administering a formulation such as a liquid formulation to a patient to relieve a symptom of Dravet syndrome.

It is a further object of the invention to supply a method of treatment for epilepsy comprising delivering one or more of the above 5-HT receptor agonists in combination with one or more of the above 5-HT receptor antagonists to a patient suffering from epilepsy; and administering a formulation such as a liquid formulation to a patient to relieve a symptom of Dravet syndrome.

It is a further object of the invention to provide a 5HT receptor agonist effective in preventing, treating or ameliorating seizures in combination with one or more additional agents effective in treating co-morbid symptoms or conditions associated with epilepsy in patients diagnosed with epilepsy; and administering a formulation such as a liquid formulation to a patient to relieve a symptom of Dravet syndrome.

In one preferred embodiment, one or more of the added agents is selected from the group consisting of an SSRI, an SNRI, and a triptan. In another preferred embodiment, the SSRI is selected from the group consisting of citalopram, escitalopram, fluoxetine, fluvoxamine, paroxetine, sertraline and combinations, salts, derivatives, fragments, and complexes thereof. In another preferred embodiment, the SNRI is selected from the group consisting of vortioxetine, imipramine, venlafaxine, desvenlafaxine, duloxetine, milnacipran, levomilnacipran and combinations, salts, derivatives, fragments, and complexes thereof. In another preferred embodiment the triptan is selected from the group consisting of almotriptan, frovatriptan, rizatriptan, sumatriptan, zolmitriptan, naratriptan and combinations, salts, derivatives, fragments, and complexes thereof.

It is an advantage of the invention that it provides for treatment of Dravet syndrome and reduction in seizures.

It is a further advantage of the invention that it provides for treatment of Dravet syndrome and reduction in seizures and associated developmental decline with reduced side effects and a better safety profile.

It is a further advantage of the invention that it provides for treatment of Dravet syndrome and reduction in seizures and associated developmental decline with reduced side effects and a better safety profile, wherein the side effects are selected from side effects related to one or more of addiction, aggression, appetite, blood pressure, cardiovascular function, emesis, heart rate, impulsivity, memory, mood, nausea nocicetion, penile erection, pupil dilation, respiration, sexual behavior, sleep, sociability, thermoregulation, vasoconstriction, learning, locomotion, migraine, anxiety, cognition, imagination, perception, GI motility.

It is a further advantage of the invention that it provides for treatment of Dravet syndrome and reduction in seizures and associated developmental decline with reduced side effects and a better safety profile, wherein the side effects are selected from cardiovascular side effects selected from pulmonary hypertension, valvulopathy, cardiac valve hypertrophy, aortic regurgitation, mitral regurgitation, lesions, and surface plaques.

It is a further advantage of the invention that it provides for treatment of Dravet syndrome and reduction in seizures and associated comorbid conditions, wherein the comorbid conditions are selected from behavioral and developmental delays, movement and balance issues, orthopedic conditions, delayed language and speech issues, growth and nutrition issues, sleeping difficulties, chronic infections, sensory integration disorders, and disruptions of the autonomic nervous system.

These and other objects, advantages, and features of the invention will become apparent to those persons skilled in the art upon reading the details of the formulations and methodology as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 consists of panels A, B, C, D, E and F which relate to: genotyping and characterization of the scn1Lab mutation.

FIG. 2 consist of panels A and B which relate to characterization of the homozygous scn1Lab−/− mutation.

FIG. 3 is a bar graph indicating that homozygous scn1Lab−/− larvae show higher locomotor activity than age-matched wildtype scn1Lab+/+.

FIG. 4 consists of images A, B, C and D which illustrate spontaneous electrographic activity from (A) 7 dpf wildtype scn1Lab+/+larva

FIG. 5 consists of bar graphs A, B and C which relate to quantification of electrographic activity underlines higher epileptiform activity in 7 dpf homozygous scn1Lab−/− larvae, compared to 7 dpf wildtype scn1Lab+/+.

FIG. 6 is a single bar graph showing long treatment (22 h) with fenfluramine 25 μM (FA) lowers locomotor activity at 7 dpf in scn1Lab−/− mutants but not in wildtype scn1Lab+/+.

FIG. 7 consists of bar graphs A, B and C and image D which relate to quantification of electrographic activity confirms the anti-epileptiform activity of fenfluramine.

FIG. 8 is a single bar graph relating to reduction in amount of neurotransmitters in 7 dpf scn1Lab−/− mutants compared to age matched wildtype scn1Lab+/+.

FIG. 9 consists of two images for short term and long term treatment relating to activity profile of agonists.

FIG. 10 consists of a single image relating to long term treatment showing activity profile of agonists.

FIG. 11 consists of two images relating to short term and long term treatment showing activity profile of fenfluramine.

FIG. 12 consists of table 1 which list of fenfluramine and its functional analogs (agonists).

FIG. 13 consists of table 2 which list of antagonists

FIG. 14 consists of table 3 which activity profile of functional analogs (agonists).

FIG. 15 consists of table 4 which shows information relating to the activity profile of fenfluramine.

FIG. 16 consists of bar graphs A & B, relating to the effects of lisuride, efavirenz and rizatriptan on locomotor activity in age-matched scn1Lab−/− mutants and wildtype scn1Lab+/+ zebrafish, as described in Example 2 herein.

FIG. 17 consists of bar graphs A, B, and C, relating to the effects of lisuride and efavirenz on epileptiform activity in age-matched scn1Lab−/− mutants and wildtype scn1Lab+/+ zebrafish, as described in Example 2 herein

FIG. 18 consists of table 5 which shows information relating to the epileptiform activity of fenfluramine, TCB-2, lorcaserin, GR 46611, BW 723C86, lisuride, and efavirenze activity in scn1Lab−/− mutants, as described in Example 2 herein.

### DETAILED DESCRIPTION OF THE INVENTION

Before the present formulations and methods are described, it is to be understood that this invention is not limited to particular formulations and methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a formulation” includes a plurality of such formulations and reference to “the method” includes reference to one or more methods and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Invention in General

As described above, fenfluramine is a 5-HT receptor agonist known to be effective in reducing seizures in patients with epilepsy, including Dravet syndrome. Fenfluramine was known to have high affinity for and activity at the 5-HT2A and 5-HT2C receptor subtypes, and it was unknown if the activity in Dravet syndrome was associated with one or more of these receptor subtypes, other 5-HT receptor subtypes, or another unrelated mode of action. Fenfluramine is primarily metabolized in humans into norfenfluramine, which has strong affinity for 5-HT2B receptors. The activation of 5-HT2B receptors is associated with cardiac valve hypertrophy. Thus the inventors were motivated to determine if it would be possible to create a treatment for Dravet syndrome that maintains the efficacy of fenfluramine while avoiding possible side effects.

Agonists with high specificity and affinity for the various 5-HT receptor subtypes were selected. Because agonists with specificity for the 5-HT5A and 5-HT5B receptors were not available, ergotamine, with affinity to both was utilized. These compounds were screened using a Zebrafish model of epilepsy utilizing both acute and chronic exposure profiles, and compared to the activity of Fenfluramine.

Fenfluramine, as expected, showed high levels of activity following both acute and chronic application, as did the 5-HT1D, 5-HT2A, 5-HT2C, 5-HT5, and 5-HT7 specific agonists.

Further, fenfluramine was used in the model in conjunction with 5-HT1D, 5-HT2A, 5-HT2C, and 5-HT7 receptor antagonists. The decrease in large movements indicative of efficacy was not seen in these trials, suggesting that the efficacy of fenfluramine is related to one or more of these receptor subtypes.

Notably, the 5-HT2B agonist did not show any activity in the Zebrafish model, demonstrating that it is possible to treat epilepsy including Dravet syndrome with 5-HT receptor agonists while avoiding the cardiac side effects associated with the 5-HT2B receptor.

5HT Receptor Agonists of the Present Disclosure

While not being bound by theory, in one aspect 5-HT receptor agonists of the disclosure herein are selective 5-HT receptor agonists having affinity to one or more of the same 5HT receptor subtypes associated with the anti-seizure effects of fenfluramine, and which show efficacy in preventing or ameliorating seizures and associated symptoms in patients diagnosed with epilepsy including Dravet syndrome, may be useful as therapeutic agents. Preferred selective 5HT receptor agonists include but not limited to selective 5-HT receptor agonists with affinity, with agonists showing high specificity to one or more of the 5HT-1D, 5HT-2A, 5HT-2C, 5HT-5A, and/or 5HT-7 being particularly preferred. In one particularly preferred exemplary embodiment of this aspect of the invention, the 5-HT selective agonist is lorcaserin due to its affinity to the 5HT-2C receptor subtype, which is 100× greater than its affinity for the 5-HT2B receptor sub-type.

Without being bound by theory, in another aspect, the 5-HT receptor agonists of the disclosure herein are selective agonists having affinity to one or more of the same 5HT receptor subtypes associated with the anti-seizure effects of fenfluramine, including but not limited to one or more of the 5HT-1D, 5HT-2A, 5HT-2C, 5HT-5A, and/or 5HT-7 receptor subtypes, but are not agonists of the 5-HT receptor subtypes associated with fenfluramine's adverse effects. Particularly preferred are compounds which are not agonists of the 5HT-2B, receptor. In a particularly preferred exemplary embodiment, the compound is efavirenz due to its affinity to the 5-HT2A and 5-HT2C receptor subtypes.

Without being bound by theory, in another aspect, the 5-HT receptor agonists of the present disclosure are selective agonists having affinity to one or more of the same 5HT receptor subtypes associated with the anti-seizure effects of fenfluramine, including but not limited to one or more of the 5HT-1D, 5HT-2A, 5HT-2C, 5HT-5A, and/or 5HT-7 receptor subtypes, are also antagonists of the 5-HT receptor subtypes which are associated with fenfluramine's adverse effects, particularly the 5-HT2B receptor sub-type. In a particularly preferred exemplary embodiment of this aspect of the disclosure, the compound is lisuride due to its affinity to the 5-HT2A receptor sub-type and concomitant antagonist activity at the 5-HT2B receptor subtype.

Dosing

By weight: The different 5-HT receptor agonists may be dosed to patients in different amounts depending on different patient age, size, sex, condition as well as the use of different 5-HT receptor agonists. However, in general the 5-HT receptor agonists use in connection with the treating of epilepsy in particular Dravet syndrome are used in substantially smaller amounts as compared to amounts in connection with the treatment of obesity. These smaller amounts may be half the dosing, one quarter of the dosing, or one tenth of the dosing used in connection with the treatment of obesity.

Daily Dosing: The dosing may be used in surprisingly low amounts and still be effective in eliminating seizures in Dravet syndrome patients. The dosing may be a daily dosing and may be dosing of less than about 0.8 mg/kg/day, 0.7 mg/kg/day, 0.6 mg/kg/day, 0.5 mg/kg/day, about 0.45 mg/kg/day, about 0.4 mg/kg/day, about 0.3 mg/kg/day, about 0.25 mg/kg/day or about 0.2 mg/kg/day to about 0.1 mg/kg/day, about 0.05 mg/kg/day, or about 0.01 mg/kg/day is employed. Put differently, each dose may be from a single dosage unit which may result in a dose of less than about 0.5 to about 0.01 mg/kg/day. Such a dose is less than the daily dose of fenfluramine suggested for administration to achieve weight loss.

The patient may be dosed on a daily basis using a single dosage unit which single dosage unit may be comprised of the 5-HT agonist in an amount of 30 mg or less, 20 mg or less, 10 mg or less, 5 mg or less, 2 mg or less, 1 mg or less and the dosage unit may be for oral delivery or injectable

Methods of Treating Obesity Distinguished

The methods of the invention are also distinguishable from methods used in the treatment of obesity in that the patients with epilepsy and in particular Dravet syndrome, are patients which are very young, whereas patients treated for obesity are generally older. Patients treated for obesity are generally over 20 years old and patients treated for Dravet syndrome are generally under 18, under 15, under 10, under 5, under 2 years old, under 1 year old, under 6 months old, or from 1 month to 6 months old.

Yet another difference between treating patients for obesity and treating patients for epilepsy and Dravet syndrome relates to the possible testing of the patients. Specifically, before treating patients for Dravet syndrome, it is desirable to test the patients for a genetic mutation. This is because patients without the mutation and with a different form of epilepsy could react adversely when treated with a 5-HT agonist receptor. Thus, it is desirable to test patients prior to treatment. Testing may be carried out for mutations in the SCN1A (such as partial or total deletion mutations, truncating mutations and I or missense mutations e.g. in the voltage or pore regions S4 to S6), SCN1 B (such as the region encoding the sodium channel (31 subunit), SCN2A, SCN3A, SCN9A, GABRG2 (such as the region encoding the y2 subunit), GABRD (such as the region encoding the a subunit) and I or PCDH19 genes have been linked to Dravet Syndrome.

Structural Derivatives of Known Compounds

Although specific 5-HT agonists have been disclosed and described above, other agonists might be created and tested based on known compounds. For example, derivatives of fenfluramine might be used to create a formulation. Such a formulation could be described as follows:

A formulation, comprising:

a pharmaceutically acceptable carrier; and

a therapeutically effective amount of a compound represented by the structure (I):

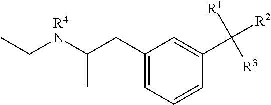

wherein R4 is selected from a group consisting of hydrogen and an alkyl group comprising one to four carbons; and each of R1, R2 and R3 are selected from the group consisting of F, Cl, Br, and I;

with the proviso that each of R1, R2 and R3 are not simultaneously F where R4 is hydrogen.

The invention includes methods of treatment and use of a formulation of a compound of formula I with a carrier to treat and relieve a symptom of epilepsy or Dravet syndrome by administering the formulation to a patient which may be administered in a liquid form.

Combination Therapy (SSRI)

The present invention includes a combination therapy whereby a 5-HT receptor agonist is combined with a selective serotonin reuptake inhibitor (SSRI). Specifically, the method includes reducing seizures in a patient with a form of epilepsy wherein the method comprises administering to the patient a therapeutically effective amount of formulation comprising a pharmaceutically acceptable carrier, a 5-HT receptor agonist, and a selective serotonin reuptake inhibitor (SSRI).

The method includes the co-administration of such drugs to treat specific forms of epilepsy such as Dravet syndrome and includes the combination of fenfluramine with an SSRI.

The 5-HT receptor agonist may be a compound which has affinity and activity at a receptor selected from the group consisting of 5-HT1D, 5-HT2A, 5-HT2C, 5-HT5A, 5-HT5B, 5-HT7 and combinations thereof. More preferably the compound is one which has affinity and activity at a receptor selected from the group consisting of 5-HT1D, 5-HT2A, 5-HT2C, 5-HT5A, 5-HT5B, 5-HT7 and combinations thereof, but that does not have affinity and activity at a receptor associated with adverse effects, particularly the 5-HT2B receptor, or is a 5-HT2B receptor antagonist.

The SSRI can be any SSRI compound such as a SSRI selector from the group consisting of citalopram, escitalopram, fluoxetine, fluvoxamine, paroxetine, sertraline and combinations, salts, derivatives, fragments, and complexes thereof.

Dosing of the 5-HT receptor agonist can be as indicated above. The dosing of the SSRI compound can be in amounts in the range of 1 mg to 50 mg administered once per day, twice per day, three times per day, or four times per day. The dosage amount may be in any incremental amount between 1 mg once a day to 50 mg four times a day and may be 1 mg, 5 mg, 10 mg, 20 mg, 30 mg, etc. administered once a day, twice a day, three times a day, four times a day, etc.

Combination Therapy (SNRI)

The present invention includes a combination therapy whereby a 5-HT receptor agonist is combined with a selective serotonin reuptake inhibitor (SNRI). Specifically, the method includes reducing seizures in a patient with a form of epilepsy wherein the method comprises administering to the patient a therapeutically effective amount of formulation comprising a pharmaceutically acceptable carrier, a 5-HT receptor agonist, and a selective serotonin reuptake inhibitor (SNRI).

The method includes the co-administration of such drugs to treat specific forms of epilepsy such as Dravet syndrome and includes the combination of fenfluramine with an SNRI.

The 5-HT receptor agonist may be a compound which has affinity for or inactivity at a receptor selected from the group consisting of 5-HT1D, 5-HT2A, 5-HT2C, 5-HT5A, 5-HT5B, 5-HT7 and combinations thereof. More preferably the compound is one which has affinity and activity at a receptor selected from the group consisting of 5-HT1D, 5-HT2A, 5-HT2C, 5-HT5A, 5-HT5B, 5-HT7 and combinations thereof, but that does not have affinity and activity at a receptor associated with adverse effects, particularly the 5-HT2B receptor, or is a 5-HT2B receptor antagonist.

The SNRI can be any SNRI compound such as a SSRI selector from the group consisting of vortioxetine, imipramine, venlafaxine, desvenlafaxine, duloxetine, milnacipran, levomilnacipran and combinations, salts, derivatives, fragments, and complexes thereof.

Dosing of the 5-HT receptor agonist can be as indicated above. The dosing of the SNRI compound can be in amounts in the range of 1 mg to 50 mg administered once per day, twice per day, three times per day, or four times per day. The dosage amount may be in any incremental amount between 1 mg once a day to 50 mg four times a day and may be 1 mg, 5 mg, 10 mg, 20 mg, 30 mg, etc. administered once a day, twice a day, three times a day, four times a day, etc.

Combination Therapy (Triptan)

The present invention includes a combination therapy whereby a 5-HT receptor agonist is combined with a triptan. Specifically, the method includes reducing seizures in a patient with a form of epilepsy wherein the method comprises administering to the patient a therapeutically effective amount of formulation comprising a pharmaceutically acceptable carrier, a 5-HT receptor agonist, and a selective serotonin reuptake inhibitor (SSRI).

The method includes the co-administration of such drugs to treat specific forms of epilepsy such as Dravet syndrome and includes the combination of fenfluramine with triptan.

The 5-HT receptor agonist may be a compound which has affinity for inactivity at a receptor selected from the group consisting of 5-HT1D, 5-HT2A, 5-HT2C, 5-HT5A, 5-HT5B, 5-HT7 and combinations thereof. More preferably the compound is one which has affinity and activity at a receptor selected from the group consisting of 5-HT1D, 5-HT2A, 5-HT2C, 5-HT5A, 5-HT5B, 5-HT7 and combinations thereof, but that does not have affinity and activity at a receptor associated with adverse effects, particularly the 5-HT2B receptor, or is a 5-HT2B receptor antagonist.

The SSRI can be any SSRI compound such as a SSRI selector from the group consisting of almotriptan, frovatriptan, rizatriptan, sumatriptan, zolmitriptan, naratriptan and combinations, salts, derivatives, fragments, and complexes thereof.

Dosing of the 5-HT receptor agonist can be as indicated above. The dosing of the triptan compound can be in amounts in the range of 1 mg to 50 mg administered once per day, twice per day, three times per day, or four times per day. The dosage amount may be in any incremental amount between 1 mg once a day to 50 mg four times a day and may be 1 mg, 5 mg, 10 mg, 20 mg, 30 mg, etc. administered once a day, twice a day, three times a day, four times a day, etc.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Zebrafish embryos (Danio rerio) heterozygous for the scn1Lab mutation (scn1Lab+/−) were backcrossed with Tupfel longfin wildtype (WT scn1Lab+/+). Adult zebrafish were housed at 28.0° C., on a 14/10 hour light/dark cycle under standard aquaculture conditions. Fertilized eggs were collected via natural spawning. Anaesthetized fish (tricaine 0.02%) were fin-clipped and genotyped by PCR. After genotyping, samples were purified (MinElute PCR Purification Kit) and sequenced by LGC Genomics. Age-matched Tupfel longfin wildtype larvae were used as control group (WT scn1Lab+/+). These embryos and larvae were kept on a 14/10 hour light/dark cycle in embryo medium (Danieaus): 1.5 mM HEPES, pH 7.6, 17.4 mM NaCl, 0.21 mM KCl, 0.12 mM MgSO4, and 0.18 mM Ca(NO3)2 in an incubator at 28.0° C. All zebrafish experiments carried out were approved by the Ethics Committee of the University of Leuven (Ethische Commissie van de KU Leuven, approval number (061/2013) and by the Belgian Federal Department of Public Health, Food Safety & Environment (Federale Overheidsdienst Volksgezondheid, Veiligheid van de Voedselketen en Leefmileu, approval number LA1210199).

To evaluate the locomotor activity of homozygous scn1Lab−/− mutants and control WT scn1Lab+/+, zebrafish larvae were placed in a 96-well plate in 100 μL of embryo medium from 4 to 8 dpf. Each day the larvae were tracked in an automated tracking device (ZebraBox™ apparatus; Viewpoint, Lyon, France) for 10 min after 30 min habituation (100-second integration interval). All recordings were performed at the same time during daytime period. The total distance in large movements was recorded and quantified using ZebraLab™ software (Viewpoint, Lyon, France). Data were pooled together from at least three independent experiments with at least 24 larvae per condition.

Epileptiform activity was measured by open-field recordings in the zebrafish larval forebrain at 7 dpf. Homozygous scn1Lab−/− mutants and control WT scn1Lab+/+ were embedded in 2% low-melting-point agarose (Invitrogen) to position a glass electrode into the forebrain. This glass electrode was filled with artificial cerebrospinal fluid (aCSF) made from: 124 mM NaCl, 2 mM KCl, 2 mM MgSO4, 2 mM CaCl2, 1.25 mM KH2PO4, 26 mM NaHCO₃ and 10 mM glucose (resistance 1-5 MΩ) and connected to a high-impedance amplifier. Subsequently, recordings were performed in current clamp mode, low-pass filtered at 1 kHz, high-pass filtered 0.1 Hz, digital gain 10, at sampling intervals of 10 μs (MultiClamp 700B amplifier, Digidata 1440A digitizer, both Axon instruments, USA). Single recordings were performed for 10 min. Epileptiform activity was quantified according to the duration of spiking paroxysms as described previously (Orellana-Paucar et al, 2012). Electrograms were analyzed with the aid of Clampfit 10.2 software (Molecular Devices Corporation, USA). Spontaneous epileptiform events were taken into account when the amplitude exceeded three times the background noise and lasted longer than 50 milliseconds (ms). This threshold was chosen due to the less frequent observation of epileptiform events in wildtype ZF larvae with a shorter duration than 50 ms.

Fenfluramine was obtained from Peak International Products B.V. Functional analogs (agonists) and antagonists were chosen based on their high and selective affinity (except for ergotamine, see further) for the different 5-HTsubtype receptors (Ki in nanomolar range), and on their log P value (i.e. >1, expected to exhibit a good bioavailability in zebrafish larvae (Milan, 2003)). Compounds were obtained from Tocris Bioscience, except for 5-HT2A-antagonist (ketaserine), 5-HT4-agonist (cisapride) and 5-HT5A-agonist (ergotamine) that were purchased from Sigma-Aldrich. Compounds were dissolved in dimethylsulfoxide (DMSO, 99.9% spectroscopy grade, Acros Organics) and diluted in embryo medium to achieve a final DMSO concentration of 0.1% w/v, which also served as a vehicle control (VHC).

To evaluate the maximal tolerated concentration (MTC) of each compound, 6 dpf-old WT scn1Lab+/+ zebrafish larvae were incubated in a 96-well plate (tissue culture plate, flat bottom, FALCON®, USA) with different concentrations of compound or VHC at 28° C. on a 14/10 hour light/dark cycle under standard aquaculture conditions (medium was replenished daily). Each larva was individually checked under the microscope during a period of 48 hours for the following signs of toxicity: decreased or no touch response upon a light touch of the tail, loss of posture, body deformation, edema, changes in heart rate or circulation and death. The maximum tolerated concentration (MTC) was defined as the highest concentration at which no signs of toxicity were observed in 12 out of 12 zebrafish larvae within 48 hours of exposure to sample. The MTC (Tables 1 and 2) was used throughout the experimental work.

ScnlLab−/− mutants and WT scn1Lab+/+ larvae were arrayed in the same plate and treated at 6 days post fertilization (dpf) with fenfluramine (25 μM), functional analogs (at their MTC) or VHC in individual wells of a 96-well plate. After incubation at 28° C. on a 14/10 hour light/dark cycle and 30-min chamber habituation 6 and 7 dpf larvae were tracked for locomotor activity for 10 min (100-second integration interval) under dark conditions. An incubation time of 1.5 hours is further referred as short treatment (6 dpf). Furthermore these larvae were analyzed after more than 22 hours incubation (7 dpf), i.e. long treatment. The total locomotor activity was quantified using the parameter lardist and plotted in cm. Data were pooled together from two (5-HT1B-, 5-HT1F-, 5-HT3-, 5-HT4-, 5-HT5A-, 5-HT6-agonist and all antagonists except 5-HT1B- and 5-HT7-antagonists) or three (fenfluramine, 5-HT1A-, 5-HT1D-, 5-HT1E-, 5-HT2A-, 5-HT2B-, and 5-HT2C-agonist) independent experiments with at least 9 larvae per treatment condition.

Epileptiform activity was measured by open-field recordings in the zebrafish larval forebrain at 7 dpf, as described above. Scn1Lab−/− mutants and WT scn1Lab+/+ larvae were incubated with fenfluramine (25 μM), the functional analogs (except for the 5-HT5A-agonist) that exhibited locomotor-reducing activity in the previous assay (see below) (MTC), a negative control (3.125 μM 5-HT2B-agonist) or VHC on 6 dpf for a minimum of 22 hours (long treatment). Recordings of 7 dpf larvae, from at least 8 scn1Lab−/− mutant larvae were taken per experimental condition. For treated WT scn1Lab+/+ larvae at least 5 per condition were analyzed, due to the scarce observation of epileptiform activity in wildtype larvae. Electrographic recordings were quantified for the different treatment conditions.

The heads of 7 dpf-old zebrafish larvae were used to determine the amount of the neurotransmitters dopamine, noradrenaline and serotonin present. Six heads per tube were homogenized on ice for one min in 100 μl 0.1 M antioxidant buffer (containing vitamin C). Homogenates were centrifuged at 15 000 g for 15 min at 4° C. Supernatants (70 μl) were transferred to a sterile tube and stored at −80° C. until analysis.

The neurotransmitter determination was based on the microbore LC-ECD method (Sophie Sarre, Katrien Thorré, Ilse Smolders, 1997) and done in collaboration with the Center for Neurosciences, C4N, VUB (Brussels, Belgium). The chromatographic system consisted of a FAMOS microautosampler of LC Packings/Dionex (Amsterdam, The Netherlands), a 307 piston pump of Gilson (Villiers-le-Bel, France), a DEGASYS DG-1210 degasser of Dionex and a DECADE II electrochemical detector equipped with a μ-VT03 flow cell (0.7 mm glassy carbon working electrode, Ag/AgCl reference electrode, 25 μm spacer) of Antec (Zoeterwoude, The Netherlands). The mobile phase was a mixture of 87% V/V aqueous buffer solution at pH 5.5 (100 mM sodium acetate trihydrate, 20 mM citric acid monohydrate, 2 mM sodium decanesulfonate, 0.5 mM disodium edetate) and 13% V/V acetonitrile. This mobile phase was injected at a flow rate of 60 μL/min. The temperature of the autosampler tray was set on 15° C. and the injection volume was 10 μL. A microbore UniJet C8 column (100×1.0 mm, 5 μm) of Bioanalytical Systems (West Lafayette, Ind., United States) was used as stationary phase. The separation and detection temperature was performed at 35° C., with a detection potential of +450 mV vs Ag/AgCl. Data acquisition was carried out by Clarity chromatography software version 3.0.2 of Data Apex (Prague, The Czech Republic). The amount of neurotransmitter (in nmol) was calculated based on the total mass of six heads.

Statistical analyses were performed using GraphPad Prism 5 software (GraphPad Software, Inc.). The larval locomotor activity was evaluated by using One-way ANOVA, followed by Dunnett's multiple comparison tests. Values were presented as means±standard deviation (SD). LFP measurements (electrographic brain activity) were analyzed by a Mann-Whitney test. Statistically significant differences (p<0.05) between a treatment group and the equivalent control groups (scn1Lab−/− mutant or WT scn1Lab+/+) were considered indicative of a decrease or increase in locomotor or electrographic brain activity of zebrafish larvae. The neurotransmitter amount of scn1Lab−/− mutants was compared with WT scn1Lab+/+ larvae by a Student's t-test because all data passed the normality test (D'Agostino & Pearson omnibus normality test).

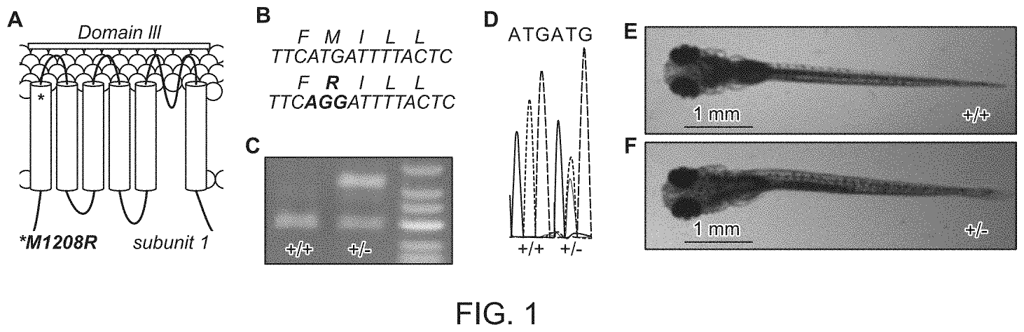

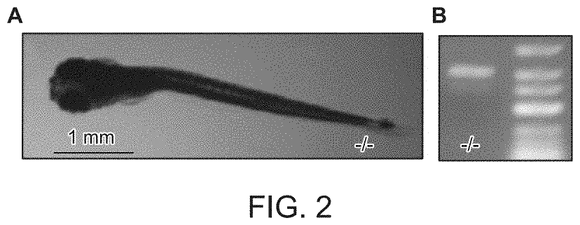

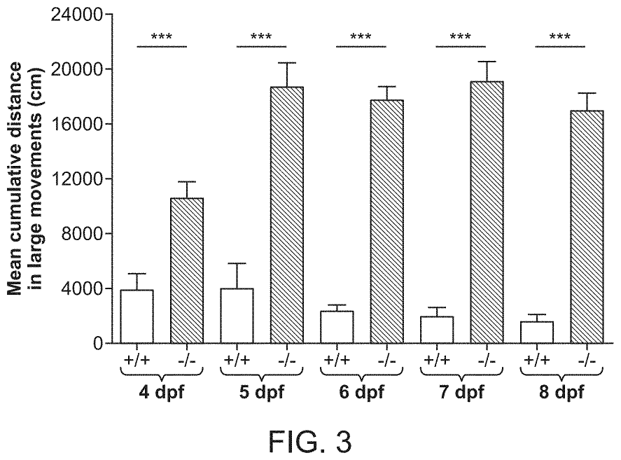

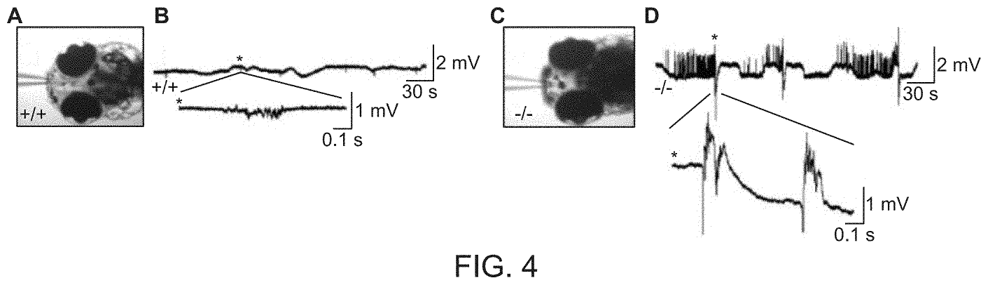

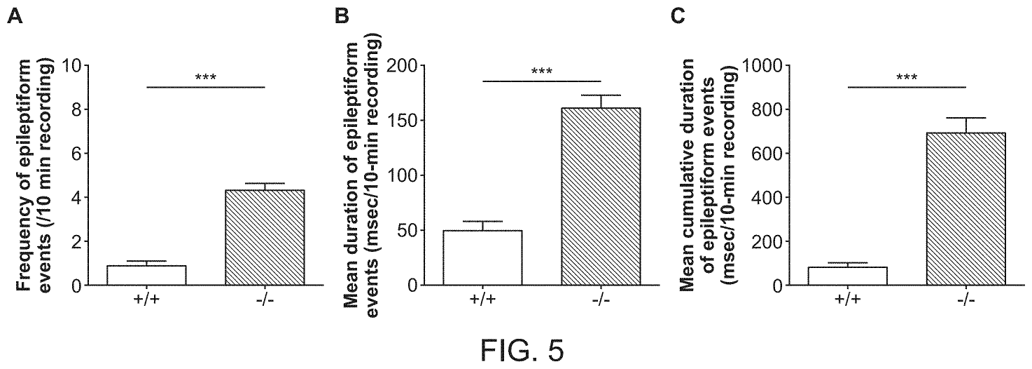

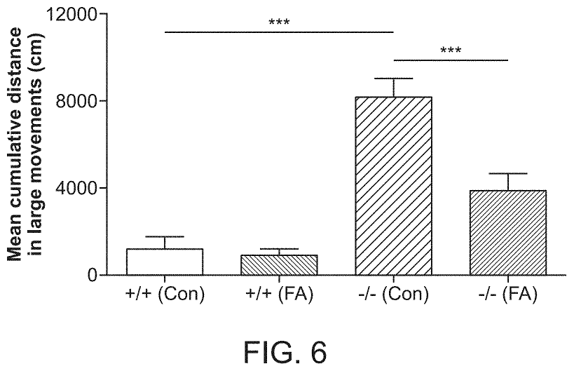

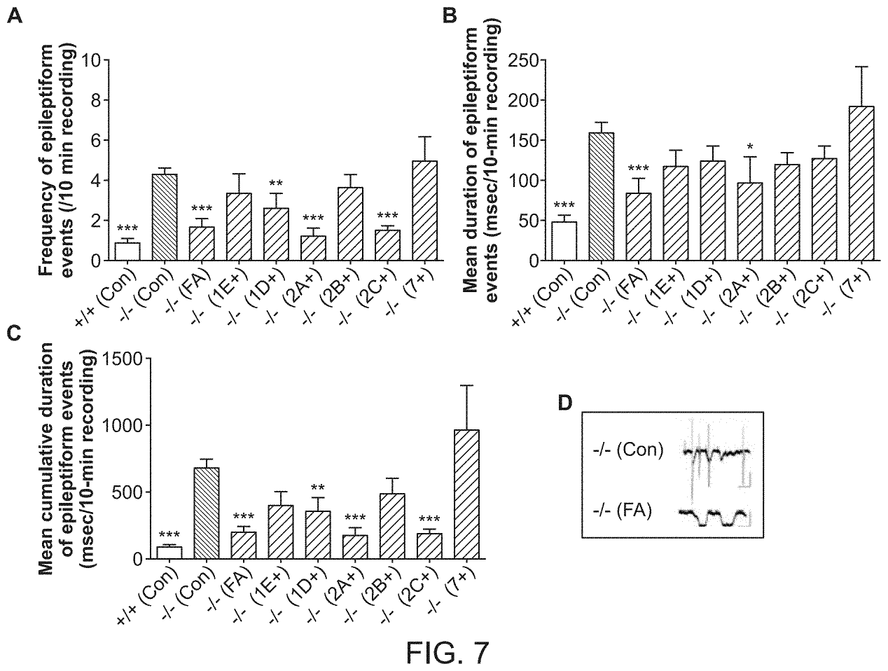

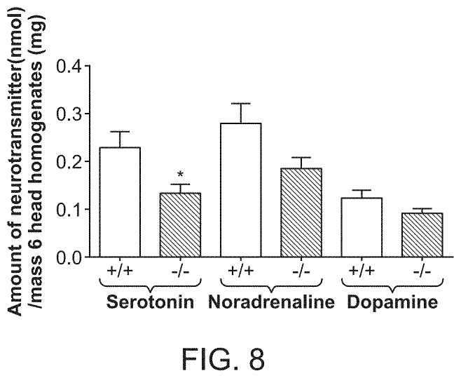

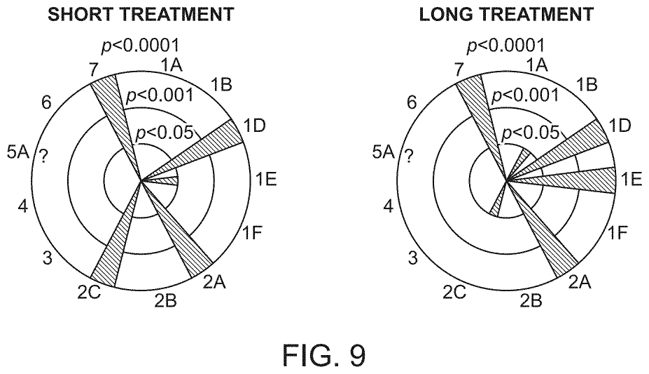

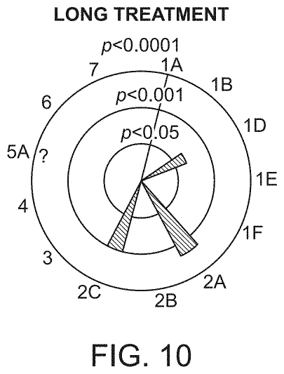

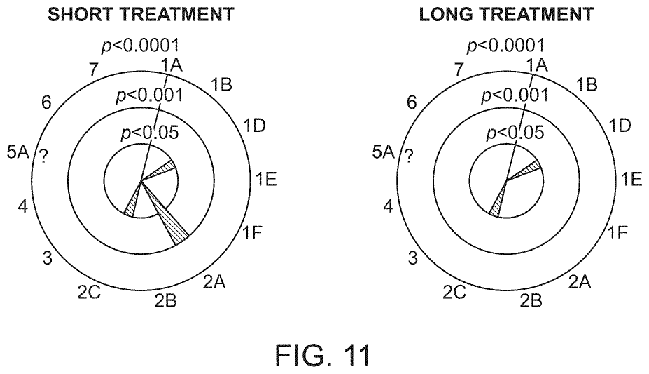

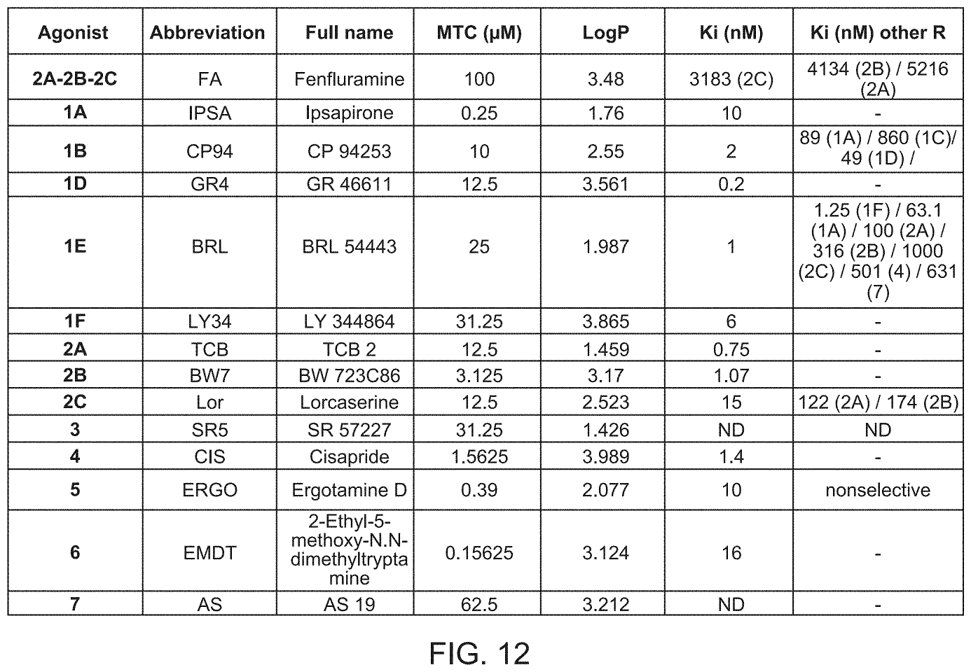

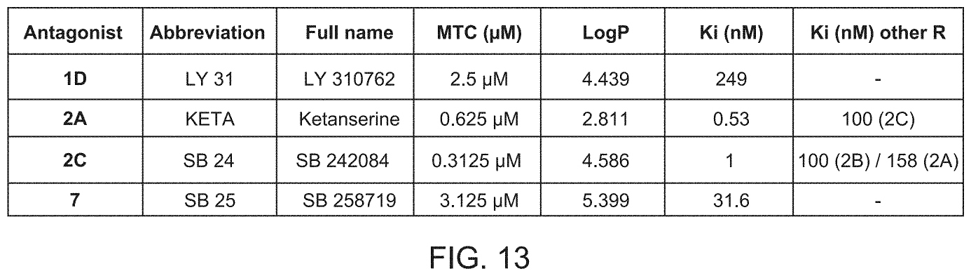

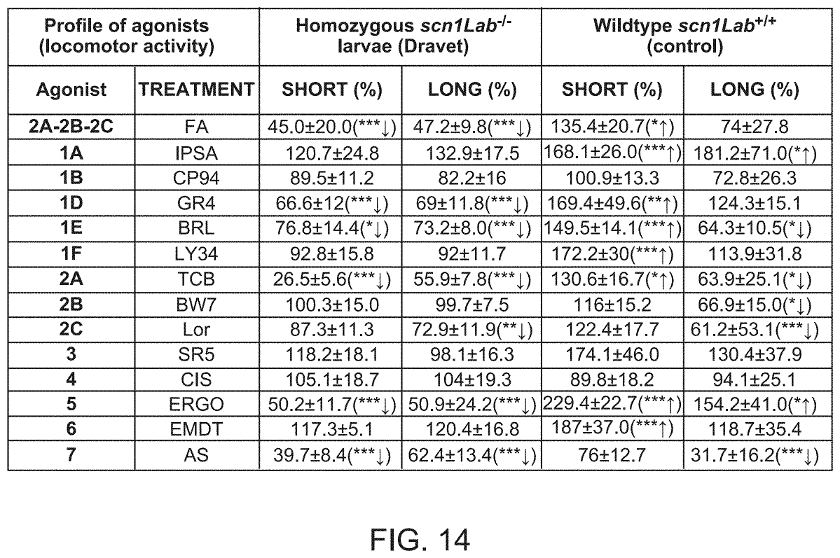

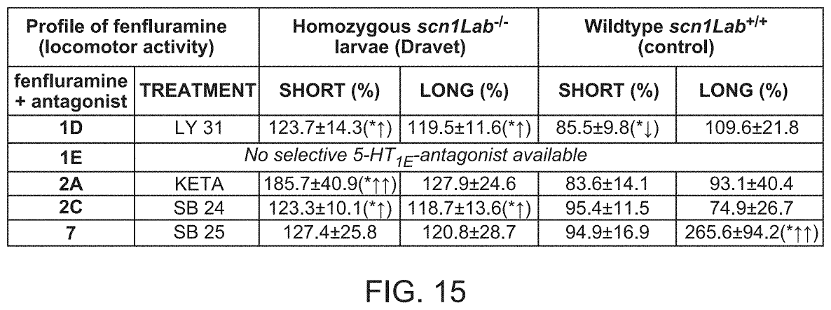

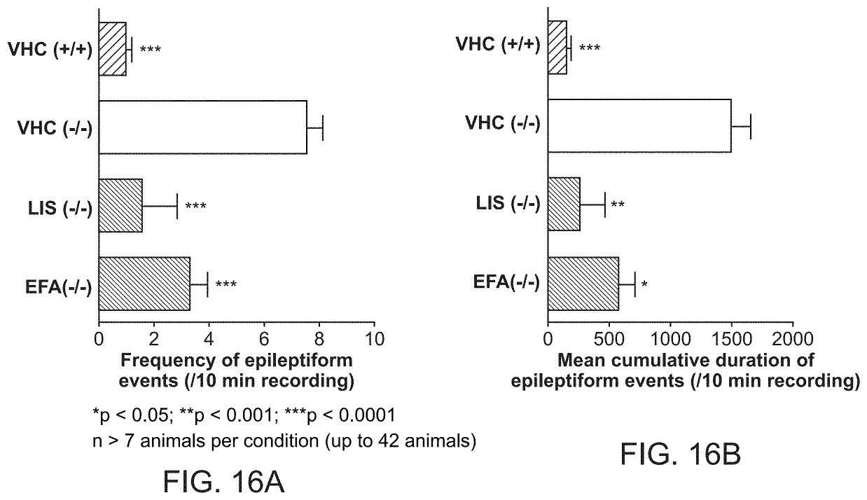

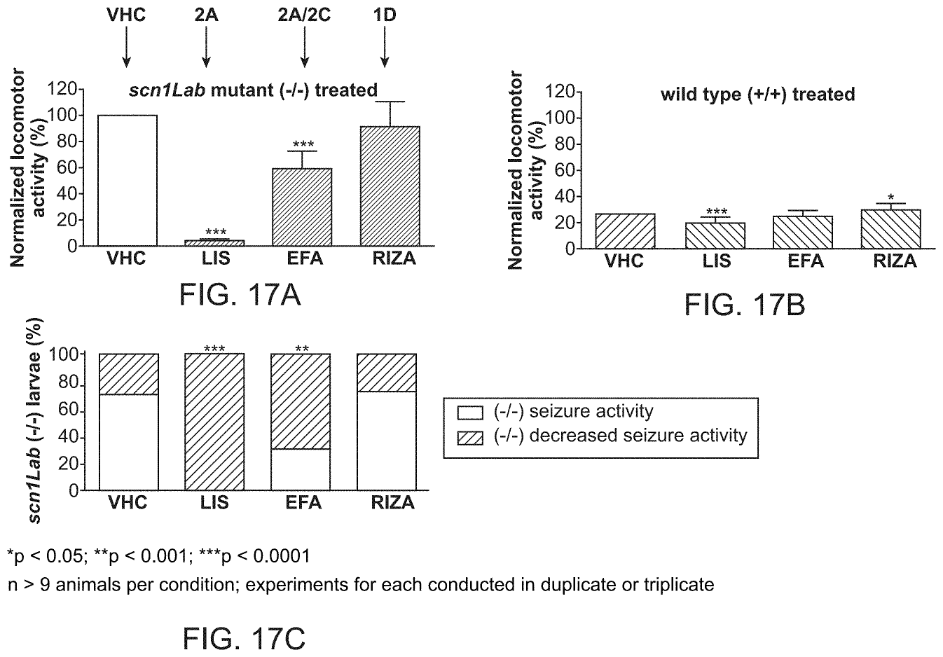

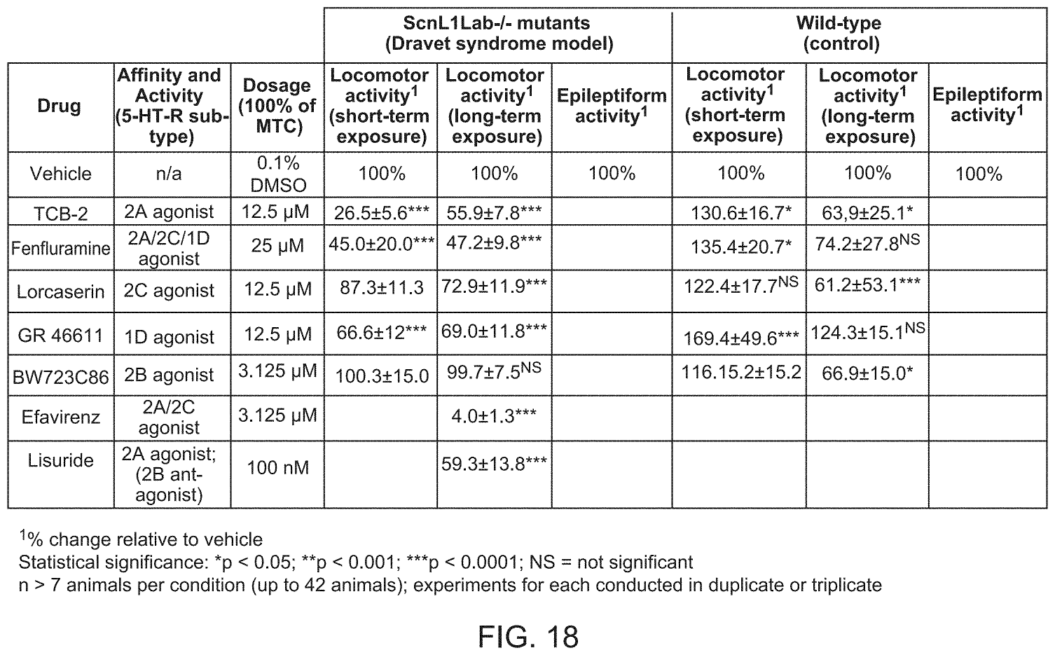

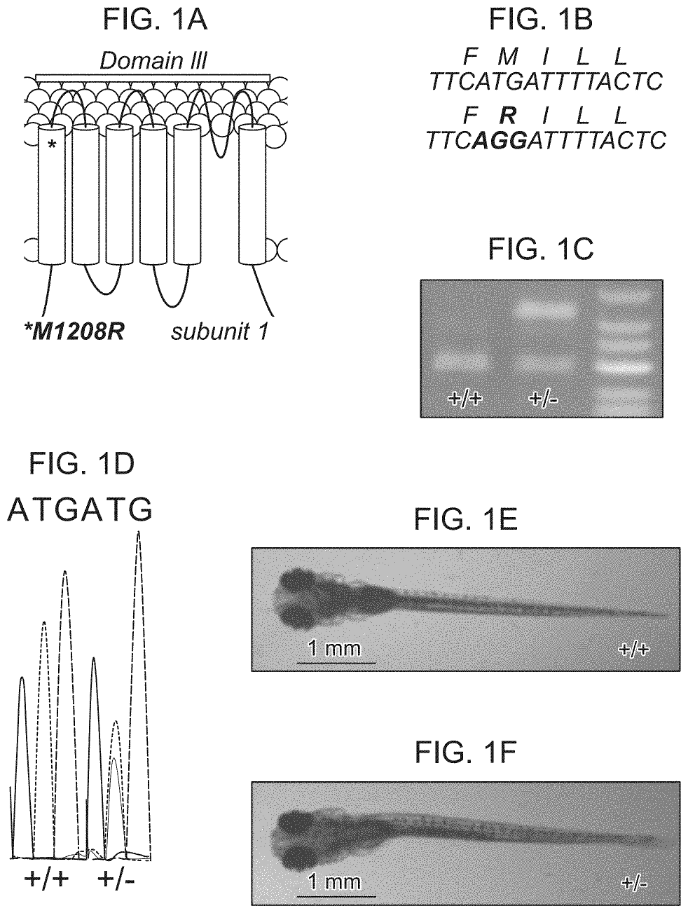

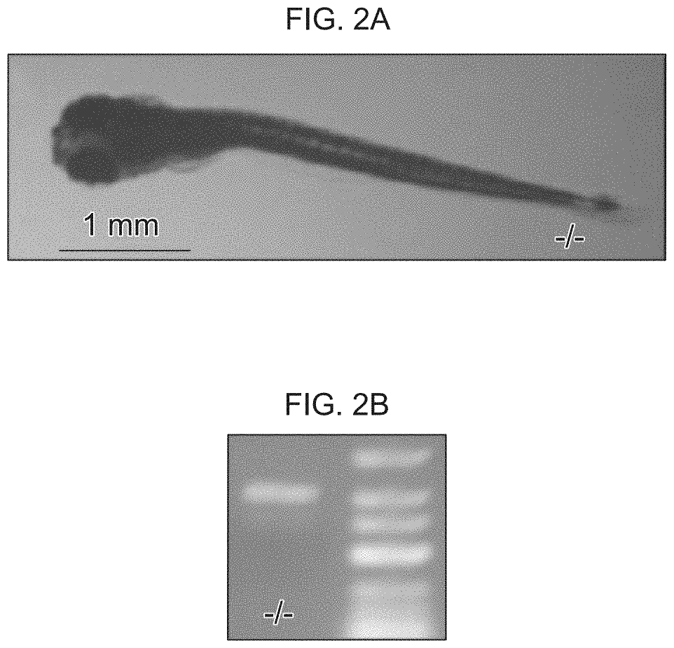

Results: The point mutation in heterozygous or homozygous scn1Lab mutants made it possible to distinguish them from WT scn1Lab+/+ by genotyping (FIG. 1). In heterozygous scn1Lab+/− mutants the PCR product contains AT3632G (wildtype allele) and AG3632G (allele with point mutation) (FIG. 1A). The point mutation converts a thymine (AT3632G) into a guanine (AG3632G), which transforms a methionine (M) to an arginine (R) (FIG. 1B). Digestion with PagI results in two fragments of different length (250 and 500 basepairs). The PCR product of adult WT scn1Lab+/+ zebrafish, on the contrary, only contains AT3632G and hence, after PagI digestion, only one fragment will be visible (250 basepairs). Homozygous scn1Lab−/− mutants solely have AG3632G. As PagI only recognizes AT3632G, genotyping of these homozygous mutants results in one visible fragment (500 basepairs). Moreover, sequencing data (LGC Genomics) confirmed the genetic difference of heterozygous scn1Lab+/− mutants (T-G mutation) compared to wildtype scn1Lab+/+. (FIG. 1D).

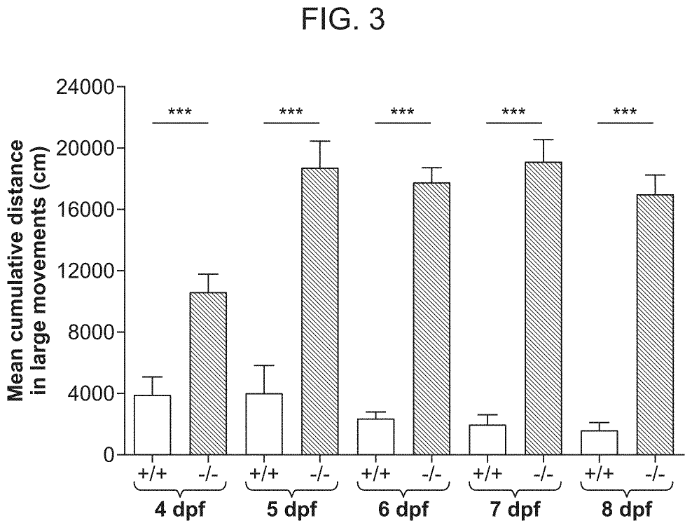

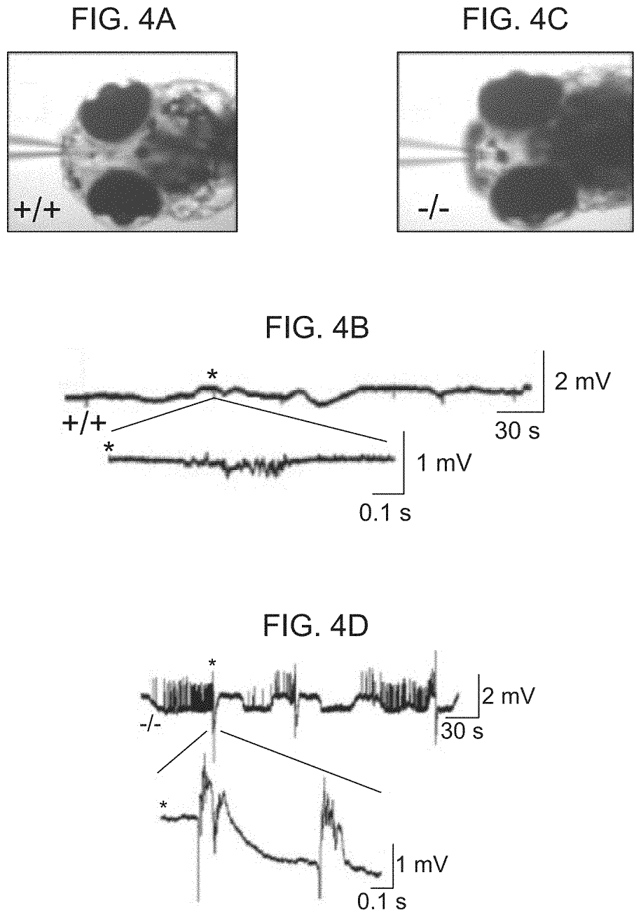

As compared to WT larvae, homozygous scn1Lab−/− mutants exhibit an increased locomotor activity expressed as total distance in large movements (lardist), thereby confirming previously published data (Baraban et al, 2013). This difference in behavior was already present at 4 dpf and was maximal between 6 dpf and 8 dpf (FIG. 3).

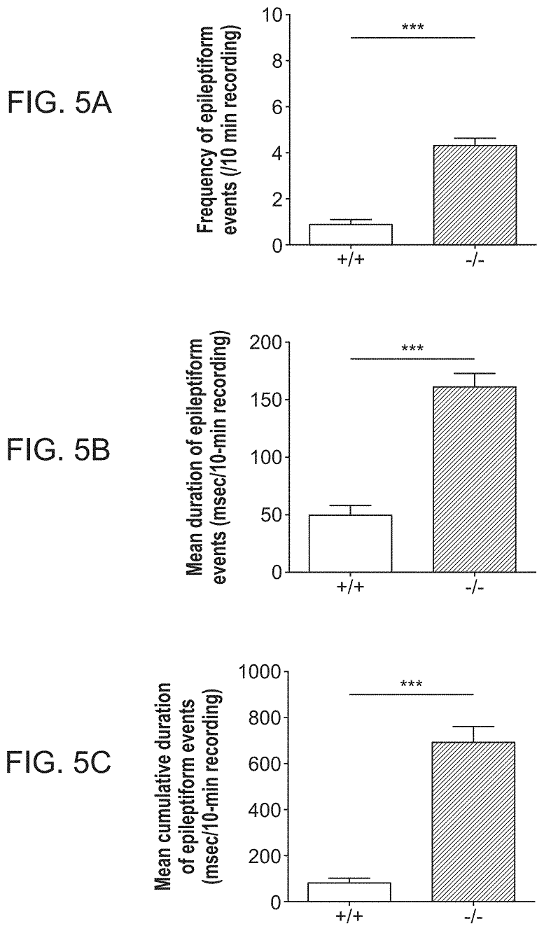

Recurrent epileptiform events happened in scn1Lab−/− mutants at a mean frequency of 4.31±0.33 events/10-min and in age-matched WT scn1Lab+/+ larvae at 0.91±0.19 events/10-min recording). This difference in frequency of epileptiform events was statistically significant (FIG. 5A, p<0.0001). As a result, the mean cumulative duration of epileptiform events was significantly higher in scn1Lab−/− mutants, compared to WT scn1Lab+/+ larvae (scn1Lab−/− mutants, 692.0±69.18 vs WT scn1Lab+/+89.62±20.33 ms/10 min-recording) (FIG. 5B, p<0.0001). Furthermore this difference was reflected in the mean duration of epileptiform events, i.e. the fraction of time spent in epileptic activity (scn1Lab−/− mutants, 160.2±11.81 vs WT scn1Lab+/+48.88±8.807 ms/10 min-recording) (FIG. 5C, p<0.0001)

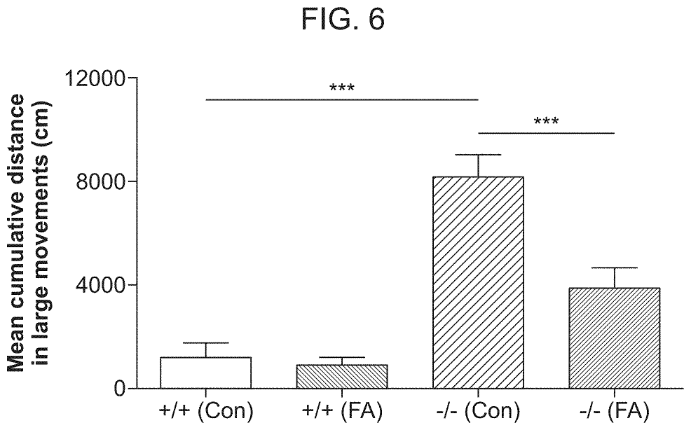

Long term treatment (22 h) with fenfluramine (25 μM) significantly decreased epileptiform locomotor activity in homozygous scn1Lab−/− mutants at 7 dpf (FIG. 6, p<0.0001). A short incubation (1.5 h) gave similar results (data not shown). Also six functional analogs of fenfluramine, i.e. 5-HT1D-, 5-HT1E-, 5-HT2A-, 5-HT2C-, 5-HT5A- and 5-HT7-agonists exhibited locomotor-reducing activity (in most cases observed after short and long treatment). Although ergotamine showed interesting activity, the compound is not a very selective 5-HT5A-agonist, and therefore this result should be intepreted with some caution. Unfortunately, no other more selective 5-HT5A-agonists are commercially available. Moreover, with exception of the 5-HT2C-, and 5-HT7-agonists, these compounds did not decrease the locomotor activity in age-matched wildtype zebrafish larvae, pointing to a selective effect on scn1Lab−/− mutants (Table 3). We also explored the locomotor-modifying activity of the 5-HT antagonists on the homozygous scn1Lab−/− mutants. However, none of them were active (data not shown), underlining the favorable effects of stimulating (instead of blocking) certain serotonin receptors on the locomotor activity of scn1Lab−/− mutants.

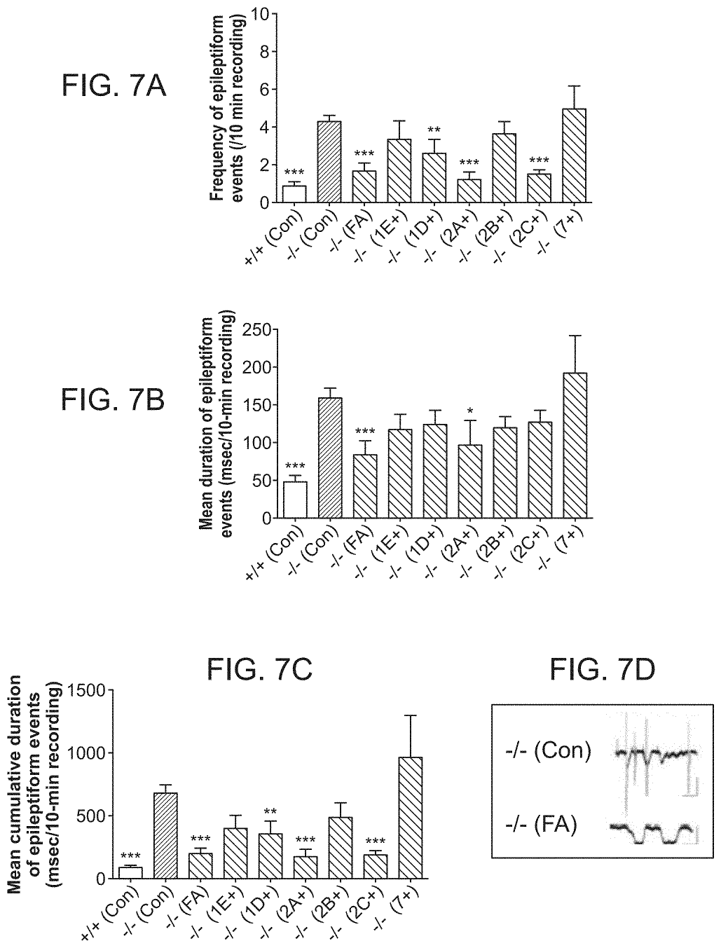

The reduction of the epileptiform activity was measured by open-field recordings in the zebrafish larval forebrain at 7 dpf after long treatment using fenfluramine and the functional analogs (except for the 5-HT5A-agonist) that exhibited locomotor-reducing activity in the previous assay. The 5-HT2B agonist was included as a negative control. Fenfluramine dramatically decreased frequency, mean cumulative duration and mean duration of epileptiform events in homozygous scn1Lab−/− mutants (respectively 1.7±0.4046 events/10-min recording; 200.8±50.38 ms/10-min recording; 85.11±18.28 ms/10-min recording) (FIG. 7). In general comparable effects were observed with the 5-HT1D-, 5-HT2C-, and especially the 5-HT2A-agonist, at least in case of the frequency and mean cumulative duration of epileptiform events (FIGS. 7A and 7C), but not with the 5-HT1E-, 5-HT7- and the 5-HT2b-agonists, in the latter case as expected (FIG. 7).

In order to explore the mechanism of action of fenfluramine, we combined the compound with antagonists of 5-HT subtype receptors that showed to be involved in the reduction of the locomotor activity as observed before, i.e. 5-HT1D-,5-HT2A-, 5-HT2C- and 5-HT7.agonists 5-HT5A was not included for reasons discussed previously. No highly specific 5-HT1E-antagonist is currently available so it was impossible to explore this 5-HTsubtype receptor in the current assay (Leung, 2009). However, earlier results showed that the 5-HT1E-agonist was not able to reduce epileptiform activity in LFP measurements, so it is unlikely that this receptor is involved in the anti-epileptiform effect of fenfluramine.

Treatment with a 5-HT1D-antagonist or a 5-HT2C-antagonist, counteracted significantly the decrease in locomotor activity as elicited by fenfluramine in scn1Lab−/− larvae, both after a short and long treatment (p<0.05) (Table 4). A similar result was seen with the 5-HT2A-antagonist, but not after a long treatment (Table 3).

Furthermore there was no inhibition of the effect of fenfluramine by the 5-HT7-antagonist. Moreover, with exception of the 5-HT7-agonist, in general these compounds had no effect in age-matched wildtype zebrafish larvae (Table 3).

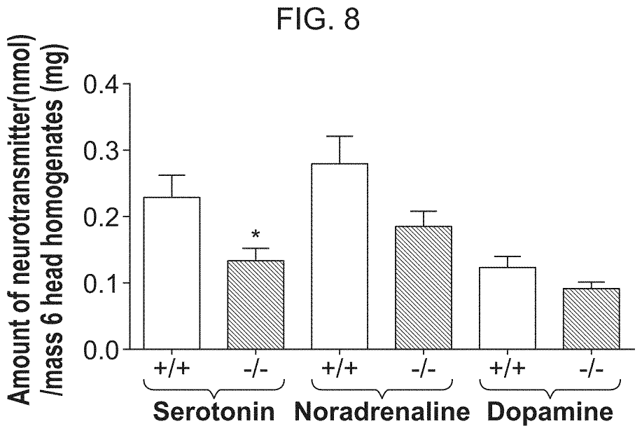

Since the previous results underline the beneficial effect of serotonergic agonists in scn1Lab−/− mutant larvae, it is likely that these DS zebrafish have an impaired neurotransmission. Hence we determined the amount of neurotransmitters in scn1Lab−/− mutants, compared to age-matched wildtype zebrafish larvae. The head homogenates of 7 dpf scn1Lab−/− mutants showed a statistically significant decrease in serotonin when compared to age-matched WT scn1Lab+/+ larvae (Student's t-test, p<0.05). There was also a decrease in the amount of dopamine and noradrenaline, but this did not reach statistically significance (Student's t-test, respectively p=0.1150; p=0.0772) (FIG. 8).

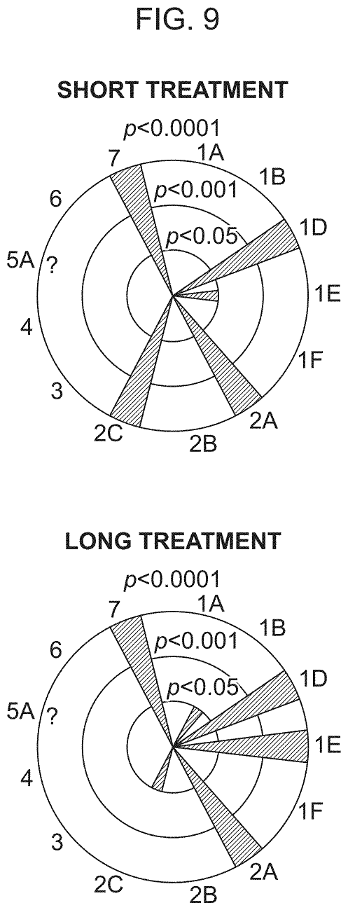

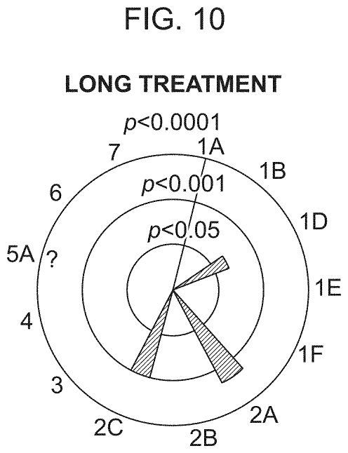

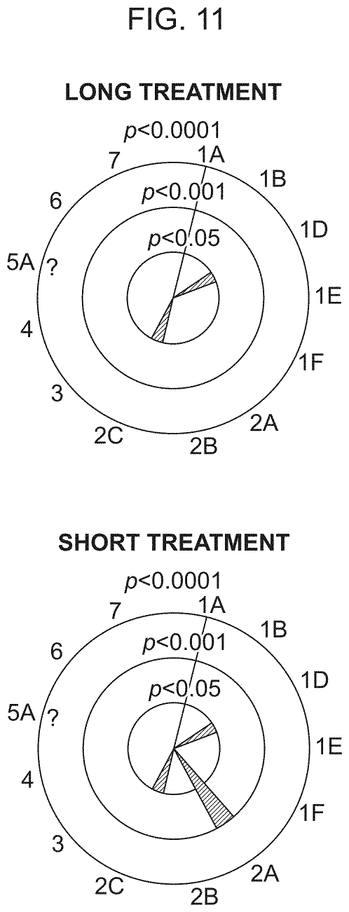

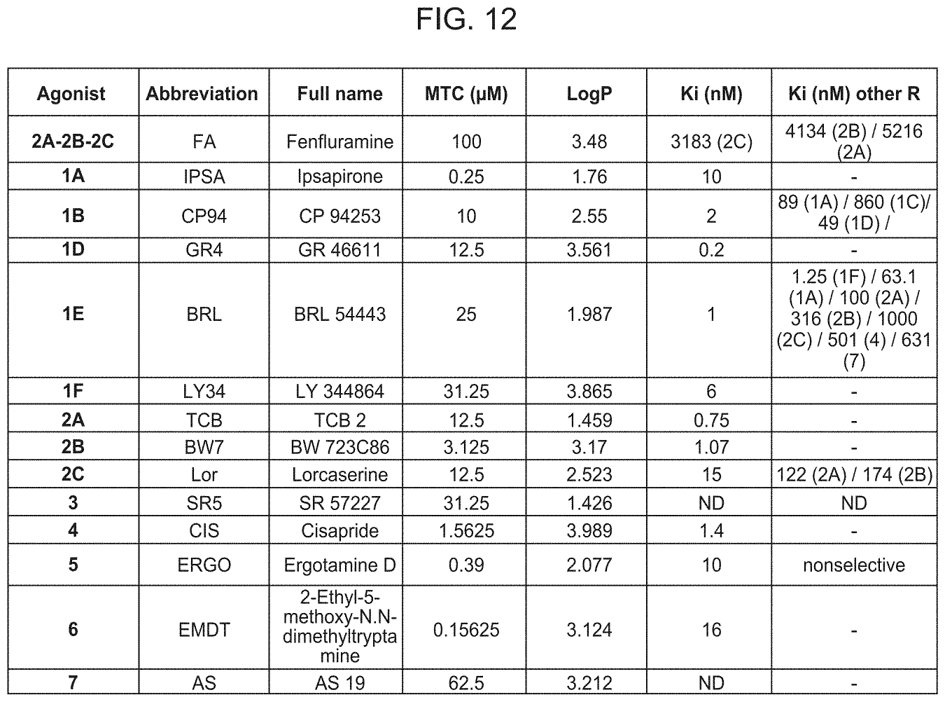

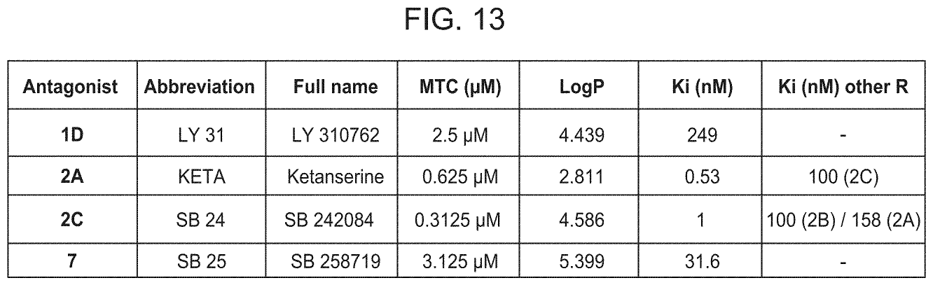

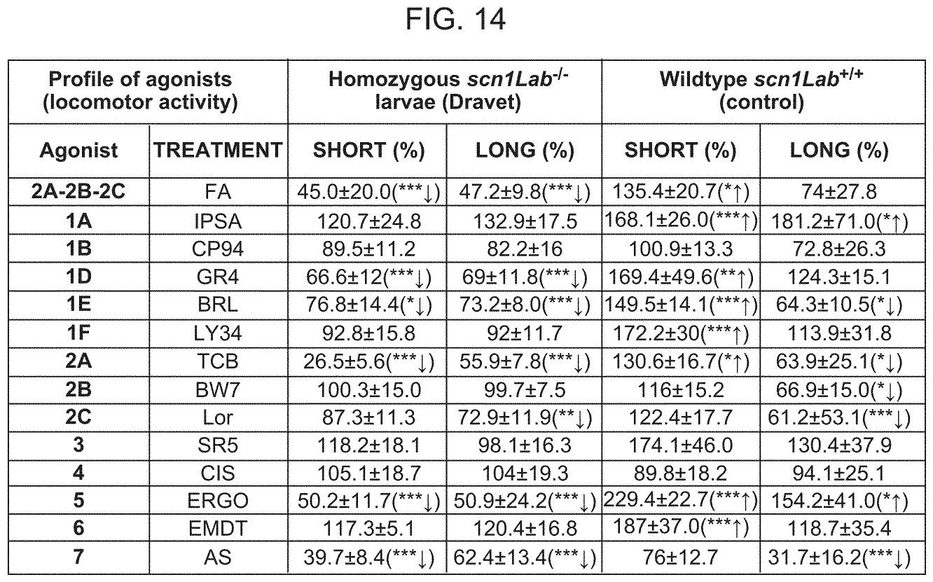

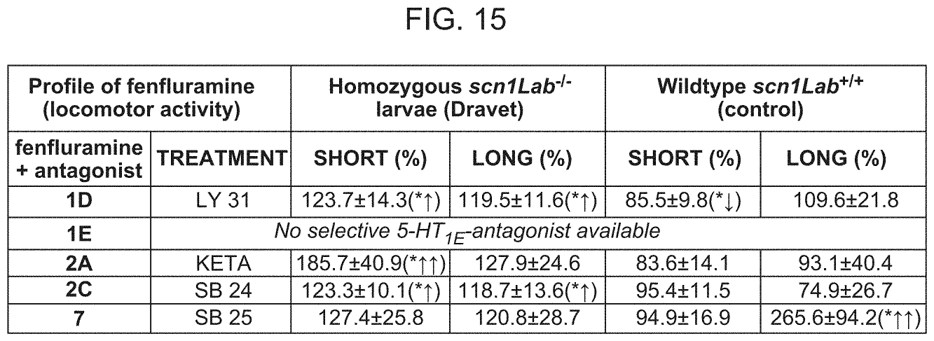

FIG. 9, 10, 11 present summaries of the activity profiles of the 5-HTsubtype agonists and fenfluramine, as depicted by radar presentations. The length of the spokes represent the statistical significance of the activity observed.

### Example 2

The drugs lisuride and efavirenz are known 5HT receptor subtype agonists. Efavirenze agonizes both the 5HT-2A and the 5HT-2C receptor subtypes [See, Gatch et al., “The HIV antiretroviral drug efavirenze has LSD-like properties” in Neuropsychopharmacology 38, pp 2373-2384 (2013)]. Lisuride agonizes the 5HT-2A receptor [See Zweckberger et al., “Anticonvulsant effects of the dopamine agonist lisuride maleate after experimental traumatic brain injury” in Neurosci. Lett. 470 pp 150-154 (2010)]; in addition, it antagonizes 5HT-2B receptor activity associated with cardio-toxic effects [See, Hofman et al., “Lisuride, a dopamine receptor agonist with 5-HT2B receptor agonist properties: absence of cardiac valvulopathy adverse drug reaction reports supports the concept of a crucial role for 5-HT2B receptor agonism in cardiac valvular fibrosis” in Clin. Neurpharmacol. 29 pp 80-86 (2006)].

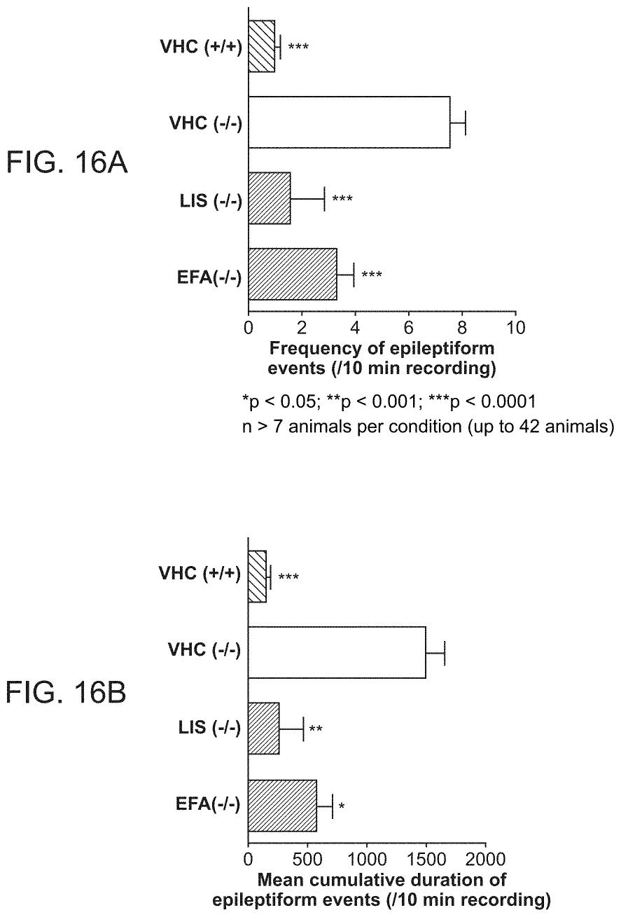

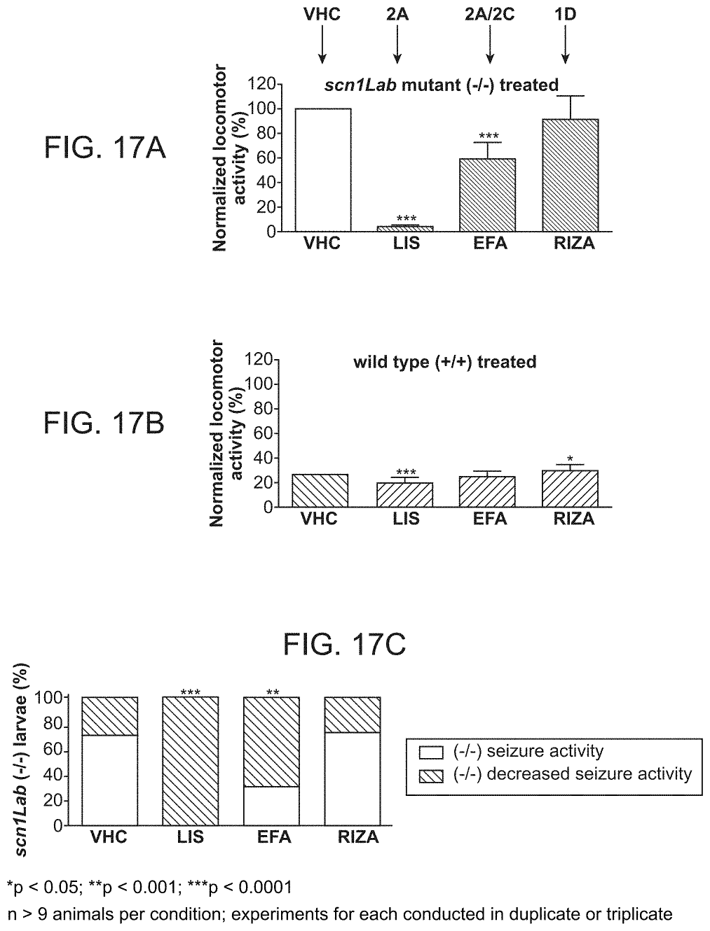

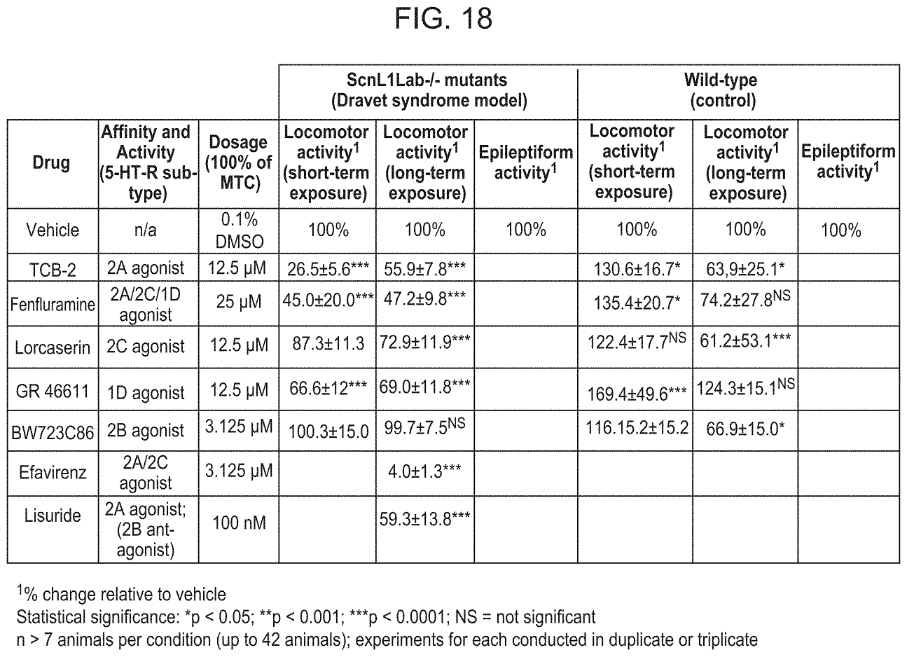

In order to identify agents with potential efficacy for treating Dravet syndrome, the effects of the 5HT receptor agonists lisuride, efavirenz, lorcaserin, TCB-2 and GR46611 on locomotion and epileptiform activity in age-matched homozygous mutant and wildtype zebrafish were investigated, as described in Example 1 above, and compared with the effects observed for fenfluramine. BW-72C86 (a 5HT-2B receptor selective agonist) and 0.1% DMSO served as negative controls. The MTC of each compound were first deteremined (data not shown) as described above. Each was then tested at its MTC in fish embryos. Results of those experiments are shown in FIGS. 16, 17, and 18.

Both efavirenz and lisuride, and particularly lisuride, are potent inhibitors of both locomotor and epileptiform activity in mutant fish embryos at the concentrations tested. This activity is consistent with that of fenfluramine in nature and extent, and suggests that both efavirenz and lisuride are potential therapeutic agents for treating Dravet syndrome.

The instant invention is shown and described herein in a manner which is considered to be the most practical and preferred embodiments. It is recognized, however, that departures may be made therefrom which are within the scope of the invention and that obvious modifications will occur to one skilled in the art upon reading this disclosure.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of reducing seizures in a patient with a form of epilepsy, comprising:
administering to the patient a therapeutically effective amount of a formulation comprising:
a pharmaceutically acceptable carrier; and
a 5-HT receptor agonist
wherein the 5-HT receptor agonist is characterized by activating a 5-HT receptor other than the 5-HT2B receptor subtype.

2. The method of claim 1, wherein the 5-HT receptor agonist has affinity for and activity at a receptor selected from the group consisting of 5-HT1D, 5-HT1E, 5-HT2A, 5-HT2C, 5-HT5A, 5-HT7, and combinations thereof.

3. The method of claim 2, wherein the 5-HT receptor agonist is selected from the group consisting of
Fenfluramine [3-trifluoromethyl-N-ethylamphetamine],
GR-46611 [3-[3-(2-Dimethylaminoethyl)-1H-indol-5-yl]-N-(4-methoxybenzyl)acrylamide],
BRL-54443 [1H-Indol-5-ol, 3-(1-methyl-4-piperidinyl)],
TCB-2 [(4-Bromo-3,6-dimethoxybenzocyclobuten-1-yl)methylamine hydrobromide],
efavirenz [(4S)-6-chloro-4-(2-cyclopropylethynyl)-4-(trifluoromethyl)-1H-3,1-benzoxazin-2-one],
lisuride [3-[(6aR,9S)-7-methyl-6,6a,8,9-tetrahydro-4H-indolo[4,3-fg]quinoline-9-yl]-1,1-diethylurea],
lorcaserin [(1R)-8-chloro-1-methyl-2,3,4,5-tetrahydro-1H-3-benzazepine], ergotamine [(6aR,9R)—N-((2R,5S,10aS,10bS)-5-benzyl-10b-hydroxy-2-methyl-3,6-dioxooctahydro-2H-oxazolo[3,2-a] pyrrolo[2,1-c]pyrazin-2-yl)-7-methyl-4,6,6a,7,8,9-hexahydroindolo[4,3-fg] quinoline-9-carboxamide],
AS-19 [(2S)-(+)-5-(1,3,5-Trimethylpyrazol-4-yl)-2-(dimethylamino)tetralin],
and combinations, salts, derivatives, fragments, and complexes thereof.

4. The method of claim 3, wherein the 5-HT receptor agonist is efavirenz [(4S)-6-chloro-4-(2-cyclopropylethynyl)-4-(trifluoromethyl)-1H-3,1-benzoxazin-2-one].

5. The method of claim 3, wherein the 5-HT receptor agonist is lorcaserin [(1R)-8-chloro-1-methyl-2,3,4,5-tetrahydro-1H-3-benzazepine].

6. The method of claim 2, wherein the 5-HT receptor agonist is also an antagonist of 5-HT2B.

7. The method of claim 6, wherein the 5-HT receptor agonist is lisuride [3-[(6aR,9S)-7-methyl-6,6a,8,9-tetrahydro-4H-indolo[4,3-fg]quinoline-9-yl]-1,1-diethylurea].

8. The method of claim 1, wherein said method further comprises:
administering one or more of 5-HT receptor antagonists,
wherein said 5-HT receptor antagonist is an antagonist of a 5-HT receptor associated with one or more unwanted side effects.

9. The method of claim 8, wherein said 5HT receptor antagonist is an ant=agonist of one or more 5-HT receptor subtypes selected from the group consisting of the 5-HT2A receptor or the 5-HT2B receptor.

10. The method of any of claim 9, wherein said 5-HT receptor antagonist is an antagonist of the 5-HT2B receptor selected from the group consisting of
Lisuride [[(6aR,9S)-7-methyl-6,6a,8,9-tetrahydro-4H-indolo[4,3-fg]quinoline-9-yl]-1,1-diethylurea],
ATC 0175 [N-[cis-4-[[4-(Dimethylamino)-2-quinazolinyl]amino]cyclohexyl]-3,4-difluorobenzamide hydrochloride],
LY 266097 [1-[(2-Chloro-3,4-dimethoxyphenyl)methyl]-2,3,4,9-tetrahydro-6-methyl-1H-pyrido[3,4-b]indole hydrochloride],
LY 272015 [1-[(3,4-Dimethoxyphenyl)methy]-2,3,4,9-tetrahydro-6-methyl-1H-pyrido[3,4-b]indole hydrochloride],
RS 127445 [4-(4-Fluoro-1-naphthalenyl)-6-(1-methylethyl)-2-pyrimidinamine hydrochloride],
SB 200646 [N-(1-Methyl-1H-indol-5-yl)-N′-3-pyridinylurea],
SB 204741 [N-(1-Methyl-1H-indolyl-5-yl)-N″-(3-methyl-5-isothiazolyl)urea],
SB 206553 [3,5-Dihydro-5-methyl-N-3-pyridinylbenzo[1,2-b:4,5-b′]dipyrrole-1(2H)-carboxamide hydrochloride],
SB 221284 [2,3-Dihydro-5-(methylthio)-N-3-pyridinyl-6-(trifluoromethyl)-1H-indole-1-carboxamide],
SB 228357 [N-[3-Fluoro-5-(3-pyrindyl)phenyl]-2,3-dihydro-5-methoxy-6-(trifluoromethyl)-1H-indole-1-carboxamide], and
SDZ SER 082 [(+)-cis-4,5,7a,8,9,10,11,11a-Octahydro-7H-10-methylindolo[1,7-bc][2,6]-naphthyridine fumarate],
and combinations, salts, derivatives, fragments, and complexes thereof.

11. The method of claim 1, further comprising:
administering to the patient a therapeutically effective amount of one or more second agents effective in preventing, treating or ameliorating one or more co-morbidity conditions associated with Dravet syndrome,
wherein one or more of said second agents are selected from the group consisting of a selective serotonin reuptake inhibitor (SSRI), a selective norepinephrine reuptake inhibitor (SNR), and a triptan.

12. The method of claim 11,
wherein said selective serotonin reuptake inhibitor (SSRI) is selected from the group consisting of citalopram, escitalopram, fluoxetine, fluvoxamine, paroxetine, sertraline and combinations, salts, derivatives, fragments, and complexes thereof,
wherein said selective norepinephrine inhibitor (SNRI), selected from the group consisting of vortioxetine, imipramine, venlafaxine, desvenlafaxine, duloxetine, milnacipran, levomilnacipran and combinations, salts, derivatives, fragments, and complexes thereof,
wherein said triptanis selected from the group consisting of almotriptan, frovatriptan, rizatriptan, sumatriptan, zolmitriptan, naratriptan and combinations, salts, derivatives, fragments, and complexes thereof.

13.-19. (canceled)

20. A method of reducing seizures in a patient with Dravet syndrome, comprising:
orally administering to the patient, a therapeutically effective amount of a liquid formulation comprising:
a pharmaceutically acceptable carrier; and
lorcaserin [(1R)-8-chloro-1-methyl-2,3,4,5-tetrahydro-1H-3-benzazepine]
co-administering stiripentol; and
co-administering a formulation comprising—a selective serotonin reuptake inhibitor (S SRI).

21. The method as claimed in claim 20, wherein the SSRI is selected from the group consisting of:
citalopram, escitalopram, fluoxetine, fluvoxamine, paroxetine, sertraline and combinations, and salts thereof.

22. A method of reducing seizures in a patient with Dravet syndrome, comprising:
orally administering to the patient, a therapeutically effective amount of a liquid formulation comprising:
a pharmaceutically acceptable carrier; and
lorcaserin [(1R)-8-chloro-1-methyl-2,3,4,5-tetrahydro-1H-3-benzazepine], and
co-administering a formulation comprising citalopram.
